(19) United States
(12) Patent Application Publication
(10) Pub. No.: US 2025/0338807 A1
(43) Pub. Date: Nov. 6, 2025

(54) CLUBROOT RESISTANCE IN BRASSICA

(71) Applicant: PIONEER HI-BRED INTERNATIONAL, INC., JOHNSTON, IA (US)

(72) Inventors: SARAH ATWOOD, ANKENY, IA (US); JENNIFER BOHN, JOHNSTON, IA (US); SUNITA R. CHILAKAMARRI, WAUKEE, IA (US); IGOR FALAK, GUELPH (CA); XIUQIANG HUANG, ONTARIO (CA); SIVA S. AMMIRAJU JETTY, JOHNSTON, IA (US); SCOTT CHARLES JOBGEN, WEST DES MOINES, IA (US)

(73) Assignee: PIONEER HI-BRED INTERNATIONAL, INC., JOHNSTON, IA (US)

(21) Appl. No.: 18/557,401

(22) PCT Filed: Apr. 27, 2022
(86) PCT No.: PCT/US2022/026519

§ 371 Date: Oct. 26, 2023

Related U.S. Application Data
(60) Provisional application No. 63/181,608, filed on Apr. 29, 2021.

(51) Int. Cl. A01H 1/00; A01H 1/04

(52) U.S. Cl. CPC A01H 1/1255; A01H 1/045

(57) ABSTRACT

Provided are methods and compositions, including assays, probes and primers for identifying Brassica plants that are resistant to clubroot disease. Also provided are breeding methods for introducing a clubroot resistance phenotype into Brassica plants and/or their progeny.

### CROSS-REFERENCE TO RELATED APPLICATIONS

This application is a national phase entry under 35 U.S.C. § 371 of International Patent Application PCT/US2022/26519, filed on Apr. 27, 2022, which claims the benefit of U.S. Provisional Application No. 63/181,608 filed on Apr. 29, 2021, each of which is incorporated by reference herein in its entirety.

### REFERENCE TO SEQUENCE LISTING SUBMITTED ELECTRONICALLY

The official copy of the sequence listing is submitted electronically via EFS-Web as an ASCII-formatted sequence listing with a file named 107838substv2_ST25, created on Jun. 4, 2024 and having a size of 74,497 bytes, which is part of the specification and is herein incorporated by reference in its entirety.

### FIELD OF THE DISCLOSURE

The present disclosure relates to plants resistant to diseases, in particular to Brassica plants resistant to clubroot disease.

### BACKGROUND

Clubroot is a widespread disease that causes major economic losses and has emerged as serious threat in many Brassica growing areas globally and particularly in North America. Clubroot disease is caused by Plasmodiophora brassicae, a soil-borne, root-infecting protist pathogen and phylogenetical intermediate between a fungus and bacteria. P. brassicae infection leads to swollen roots or ‘galls’ that hijack the host water and nutrient supplies, causing wilting, death and loss of yield. Management of clubroot is challenging because of two unique attributes of P. brassicae. The organism has very short life cycles and can produce multiple generations within a season. Second, each infected gall produces billions of spores that can survive in soil for many years and, in some cases, more than 15 years. Local spread of spores can be facilitated by wet conditions, but most dispersal of the pathogen is caused by transportation of infested soil or compost, e.g., on tools, equipment, or plant material. P. brassicae has a wide host range in the Brassica family including numerous weed species.

There are currently no effective fungicides for the widespread control of clubroot. In the absence of effective chemical control options, developing sources of genetic resistance has the most potential for protecting Brassica from clubroot. Clubroot resistance, mostly qualitative and race-specific, exists in some Brassica vegetables such as rutabaga, turnips, and cabbages. including in Chinese cabbage (Yoshikawa. 1983. Japan Agricultural Research Quarterly, 17:6-11). Chinese cabbage F1 hybrids with this resistance have been shown to have good protection against clubroot, although a small number of races have been able to break through this resistance. To date, more than 10 loci have been identified that contribute to clubroot resistance, these include: CRa, CRb, CRc, CRk (Matsumoto et al. 1998. J Jpn Soc Hortic Sci 74:367-373; Piao et al. 2004. Theor Appl Genet 108:1458-1465; Sakamoto et al. 2008. Theo Appl Genet 117:759-767), Crr1, Crr2, Crr3, Crr4 (Suwabe et al. 2003. Theo Appl Genet 107:997-1002; Suwabe et al. 2006. Genetics 173:309-319; Hirai et al. 2004. Theor Appl Genet 108:639-643), CRd (Pang et al. 2018. Front Plant Sci 9:822), PbBa3.1, PbBa3.2, PbBa3.3, PbBa1.1, PbBa8.1 (Chen et al. 2013. PLOS ONE 8 (12): e85307), Rcr1 (Chu et al. 2014. BMC Genomics 15 (1): 1166), Rcr4, Rcr8, and Rcr9 (Yu et al. 2017. Sci Rep 7 (1): 4516).

Nonetheless different subgroups or races of clubroot pathogen have been identified that exhibit virulence against plants having loci associated with a particular clubroot resistance. Additionally, repeated plantings of Brassica plants having the same (single or multiple) clubroot resistance loci may lead to the diminution and/or complete loss of effectiveness due to selection pressure for pathogens that overcome these genetic sources of resistance. This is of particular concern when varieties with clubroot resistant loci are challenged by high pathogen loads, which increases the probability for evolving new races that are virulent even for plants having those loci. Therefore, in order to mitigate the problem of evolving pathogen resistance and to protect against a broader spectrum of pathogens, there is a need and desire to identify, introgress, and track new sources of clubroot resistance in Brassica species, particularly for the commercially significant species such as Brassica napus.

### SUMMARY OF THE DISCLOSURE

Disclosed herein are genetic marker alleles, methods, and assays for identifying and tracking clubroot resistance loci on Brassica chromosome N3. The markers, methods, and assays are based, at least in part, on discoveries generated by an extensive and intensive genetic screening effort to identify new markers and/or sources of clubroot disease. The disclosed markers are tightly linked to the resistance loci CrM3, CrS3, CrT3, CrB3, and CrN3 described herein. The disclosed markers appear to be uniquely specific to resistant donor lines disclosed herein and/or are so rare in publicly available germplasm that they have not been previously identified as being linked to clubroot resistance.

The disclosed CrM3, CrS3, CrT3, CrB3, and CrN3 markers are suitable for high-throughput marker assisted selection. In certain examples, the markers are particularly suited for the identification of loci that are rare or particularly unusual. Additionally, the disclosed markers are suitable for the identification and introgression of clubroot resistance in inbred germplasm for each of these loci on chromosome N3 and can be used to generate hybrid clubroot resistant Brassica plants and seed.

Provided is a method of identifying a Brassica plant, cell, or germplasm comprising a clubroot disease resistance locus by obtaining a sample of nucleic acid from a Brassica plant, cell, or germplasm and screening the sample for a molecular marker allele, or a haplotype of molecular marker alleles, linked to one or more of the following clubroot resistance loci: (1) CrM3 located on chromosome N3 interval flanked by and including 113.88 cM and 115.57 cM, (2) CrS3 located on chromosome N3 interval flanked by and including 113.6 cM and 116.36 cM, (3) CrT3 located on chromosome N3 interval flanked by and including 59.7 cM and 69.8 cM, (4) CrB3 located on chromosome N3 interval flanked by and including 45.67 cM and 67.79 cM, or (5) CrN3 located on chromosome N3 interval flanked by and including 62.4 cM and 77.3 cM. As disclosed herein, the CrM3 locus corresponds to physical position 24,092,908 to position 25,040,472 of chromosome 3 (Chr 3); the CrS3 locus corresponds to physical position 23,965,482 to position and 24,955,776 of Chr 3; the CrT3 locus corresponds to the physical position 14,777,622 to position 16,528,042 of Chr 3; the CrB3 locus corresponds to position 10,411,130 to position 15,959,930 of Chr 3; and the CrN3 locus corresponds to position 14,959,178 to position 17,536,263 on Chr 3 of a B. napus reference genome. Examples of single nucleotide polymorphism (SNP) markers that correspond to resistance (RES) and susceptibility (SUS) alleles for clubroot disease are identified in Tables 1-5. The probe sequences disclosed in Tables 1-5 comprises sequence flanking each of these SNPs. Many of the probe sequences (including the bolded and underlined SNP nucleotide) displayed in Tables 1-5 correspond to the genomic strand sequence complementary to that shown in the columns for the corresponding RES and SUS alleles. Thus, for every method disclosed herein for a particular SNP nucleotide or flanking maker sequence, it is understood that the disclosed method also includes the SNP nucleotide or flanking sequence, respectively, on the complementary strand.

In some examples, the method of identifying a Brassica plant, cell, or germplasm comprising a clubroot disease resistance locus comprises screening for at least one of the following molecular marker alleles (e.g., a haplotype that includes two or more of the following marker alleles): a CrM3 resistance marker allele identified in Table 1 herein; a CrS3 resistance marker allele identified in Table 2 herein; a CrT3 resistance marker allele identified in Table 3 herein; a CrB3 resistance marker allele identified in Table 4 herein; or a CrN3 resistance marker allele identified in Table 5 herein. Thus, for example, the method can include screening for a haplotype comprising (A) 2, 3, 4, 5, 6 or more resistance marker alleles in Table 1 herein; (B) 2, 3, 4, 5, 6 or more resistance marker alleles in Table 2 herein; (C) 2, 3, 4, 5, 6 or more resistance marker alleles identified in Table 3 herein; (D) 2, 3, 4, 5, 6 or more resistance marker alleles in Table 4 herein; or (E) 2, 3, 4, 5, 6 or more marker resistance alleles in Table 5 herein.

Additionally, the disclosed method can include screening for one or more CrM3 resistance alleles identified in Table 1 herein in combination with one or more CrS3 resistance allele identified in Table 2 herein, one or more CrT3 resistance allele identified in Table 3 herein, one or more CrB3 alleles identified in Table 4 herein, or one or more CrN3 resistance alleles identified in Table 5 herein. Or the method can include screening for one or more CrS3 resistance allele identified in Table 2 herein in combination with one or more CrM3 resistance alleles identified in Table 1 herein, one or more CrT3 resistance allele identified in Table 3 herein, one or more CrB3 alleles identified in Table 4 herein, or one or more CrN3 resistance alleles identified in Table 5 herein. Or the method can include screening for one or more CrT3 resistance allele identified in Table 3 herein in combination with one or more CrM3 resistance alleles identified in Table 1 herein, one or more CrS3 resistance allele identified in Table 2 herein, one or more CrB3 alleles identified in Table 4 herein, or one or more CrN3 resistance alleles identified in Table 5 herein. Or the method can include screening for one or more CrB3 alleles identified in Table 4 herein in combination with one or more CrM3 resistance alleles identified in Table 1 herein, one or more CrS3 resistance allele identified in Table 2 herein, one or more CrT3 resistance allele identified in Table 3 herein, or one or more CrN3 resistance alleles identified in Table 5 herein. Or the method can include screening for one or more CrN3 resistance alleles identified in Table 5 herein in combination with one or more CrM3 resistance alleles identified in Table 1 herein, one or more CrS3 resistance allele identified in Table 2 herein, one or more CrT3 resistance allele identified in Table 3 herein, or one or more CrB3 alleles identified in Table 4 herein.

In some examples, the method of identifying a Brassica plant, cell, or germplasm comprising a clubroot disease resistance locus comprises screening for at least one of the following resistance marker alleles: (1) N88673-001-Q001 (SEQ ID NO:10) allele linked to CrM3; (2) N100DIC-001-Q001 (SEQ ID NO:149) allele linked to CrS3; (3) N89533-001-Q001 (SEQ ID NO: 209), N0014XW-001-Q001 (SEQ ID NO:185), N001579-001-Q001 (SEQ ID NO:198), N0015HM-001-Q001 (SEQ ID NO:202), N0014YG-001-Q001 (SEQ ID NO:193), N0014Y6-001-Q001 (SEQ ID NO:190), or N0015V5-001-Q001 (SEQ ID NO:206) allele linked to CrT3; (4) N23443-001-Q001 (SEQ ID NO:213), N23448-001-Q001 (SEQ ID NO:218), N001579-001-Q003 (SEQ ID NO:222), N0015UF-001-Q001 (SEQ ID NO:226), N0015G9-001-Q001 (SEQ ID NO: 230), N0015V5-001-Q001 (SEQ ID NO:234), or N0014YY-001-Q001 (SEQ ID NO:237) allele linked to CrB3; (5) N100CP1-001-Q001 (SEQ ID NO:286) allele linked to CrN3.

Moreover, each of the methods for identifying a Brassica plant, cell, or germplasm comprising a clubroot disease resistance locus disclosed herein can further include selecting the Brassica plant, cell, or germplasm thereof based on the presence of the molecular marker allele or a haplotype of molecular marker alleles linked to the clubroot resistance locus. Thus, provided herein is a method of selecting a plant identified by any of the methods disclosed herein as having one or more CrM3 resistance allele identified in Table 1 herein; one or more CrS3 resistance allele identified in Table 2 herein; one or more CrT3 resistance allele identified in Table 3 herein; one or more CrB3 allele identified in Table 4 herein; or one or more CrN3 resistance allele identified in Table 5 herein. The disclosed selection methods are particularly useful for identifying and selecting such a Brassica plant, cell, or germplasm from a plurality (e.g., in a breeding population). Accordingly, the disclosed methods can be used for marker assisted selection and/or introgression of the CrM3, CrS3, CrT3, CrB3, and CrN3 loci disclosed herein.

For example, disclosed herein is a method of introducing (e.g., introgressing) at least one clubroot resistance locus into a Brassica plant by crossing a first parent Brassica plant comprising at least one clubroot resistance locus with a second Brassica plant to produce progeny plants, which can be screened for the presence or absence of one or more CrM3, CrS3, CrT3, CrB3, or CrN3 clubroot disease resistance locus using any of the screening methods disclosed herein. Thus progeny plants having at least one molecular marker allele or a haplotype that includes two or more of marker alleles identified in Tables 1-5 can be identified using any of the marker allele screening methods disclosed herein (optionally, such method can include screening for the presence of one or more susceptibility alleles disclosed in Tables 1-5 that corresponds to the one or more screened-for resistance alleles and removing or discarding plants having the susceptibility allele instead of the screened-for resistance allele). The introgression method can then include selecting one or more progeny plants having the CrM3, CrS3, CrT3, CrB3, or CrN3 clubroot disease resistance locus that is screened for. In particular examples, the introgression method can further include crossing the selected one or more progeny plants with the second parent Brassica plant to produce backcross progeny plants. Such backcross progeny plants can be screened for the presence or absence of the CrM3, CrS3, CrT3, CrB3, or CrN3 clubroot disease resistance marker alleles to thereby identify and select backcross progeny plants having a CrM3, CrS3, CrT3, CrB3, or CrN3 clubroot disease resistance locus. The selected backcross progeny plant can itself be backcrossed to the second parent Brassica plant to produce further backcross progeny plants, which can be screened as described to enable selection of further backcross progeny plants having a CrM3, CrS3, CrT3, CrB3, or CrN3 clubroot disease resistance locus. Such backcrossing, screening, and selection can be repeated for two, three, four, five, six or more generations to introgress the CrM3, CrS3, CrT3, CrB3, or CrN3 clubroot disease resistance locus into the genetic background of the second parent Brassica plant.

The disclosure can be more fully understood from the following detailed description and the accompanying drawings and Sequence Listing, which form a part of this application. The sequence descriptions and sequence listing attached hereto comply with the rules governing nucleotide and amino acid sequence disclosures in patent applications as set forth in 37 C.F.R. §§ 1.821 and 1.825. The sequence descriptions comprise the three letter codes for amino acids as defined in 37 C.F.R. §§ 1.821 and 1.825, which are incorporated herein by reference. When one strand of each nucleic acid sequence is shown, the complementary strand is understood to be included by any reference to the displayed strand.

### DETAILED DESCRIPTION

Terms used in the claims and specification are defined as set forth below unless otherwise specified. It must be noted that, as used in the specification and the appended claims, the singular forms “a,” “an” and “the” include plural referents unless the context clearly dictates otherwise.

### Terms and Definitions

An “allele” is one of several alternative forms of a gene occupying a given locus on a chromosome. When all the alleles present at a given locus on a chromosome are the same, that plant is “homozygous” at that locus. If the alleles present at a given locus on a chromosome differ, that plant is “heterozygous” at that locus.

An “amplicon” is amplified nucleic acid, e.g., a nucleic acid that is produced by amplifying a template nucleic acid by any available amplification method (e.g., PCR, LCR, transcription, or the like).

“Backcrossing” refers to the process whereby hybrid progeny plants are repeatedly crossed back to one of the parents. In a backcrossing scheme, the “donor” parent refers to the parental plant with the desired gene or locus to be introgressed. The “recipient” parent (used one or more times) or “recurrent” parent (used two or more times) refers to the parental plant into which the gene or locus is being introgressed. Backcrossing has been widely used to introduce new traits into plants. Sec e.g., Jensen, N., Ed. Plant Breeding Methodology, John Wiley & Sons, Inc., 1988. In a typical backcross protocol, the original variety of interest (recurrent parent) is crossed to a second variety (non-recurrent parent) that carries a gene of interest to be transferred. The resulting progeny from this cross are then crossed again to the recurrent parent, and the process is repeated until a plant is obtained wherein essentially all of the desired morphological and physiological characteristics of the recurrent plant are recovered in the converted plant, in addition to the transferred gene from the nonrecurrent parent.

“Brassica” refers to any one of Brassica napus (AACC, 2n=38), Brassica juncea (AABB, 2n=36), Brassica carinata (BBCC, 2n=34), Brassica rapa (syn. B. campestris) (AA, 2n=20), Brassica oleracea (CC, 2n=18) or Brassica nigra (BB, 2n=16).

The term “cross” (or “crossed”) refers to the fusion of gametes via pollination to produce progeny (e.g., cells, seeds, and plants). This term encompasses both sexual crosses (i.e., the pollination of one plant by another) and selfing (i.e., self-pollination, for example, using pollen and ovule from the same plant).

The term “elite line” means any line that has resulted from breeding and selection for superior agronomic performance. An elite plant is any plant from an elite line.

The term “gene” (or “genetic element”) may refer to a heritable genomic DNA sequence with functional significance. A gene includes a nucleic acid fragment that expresses a functional molecule such as, but not limited to, a specific protein, including regulatory sequences preceding (5′ non-coding sequences) and following (3′ non-coding sequences) the coding sequence, as well as intervening intron sequences. The term “gene” may also be used to refer to, for example and without limitation, a cDNA and/or an mRNA encoded by a heritable genomic DNA sequence.

The term “genome” as it applies to a prokaryotic and eukaryotic cell or organism cells encompasses not only chromosomal DNA found within the nucleus, but organelle DNA found within subcellular components (e.g., mitochondria, or plastid) of the cell.

A “genomic sequence” or “genomic region” is a segment of a chromosome in the genome of a cell that is present on either side of the target site or, alternatively, also comprises the target site or a portion thereof. An “endogenous genomic sequence” refers to genomic sequence within a plant cell.

As used herein, “gene” includes a nucleic acid fragment or sequence that expresses a functional molecule such as, but not limited to, a specific protein coding sequence and regulatory elements, such as those preceding (5′ non-coding sequences) and following (3′ non-coding sequences) the coding sequence.

A “genomic locus” as used herein refers to the genetic or physical location on a chromosome of a gene.

The term “genotype” refers to the physical components, i.e., the actual nucleic acid sequence at one or more loci in an individual plant.

The term “germplasm” refers to genetic material of or from an individual plant or group of plants (e.g., a plant line, variety, and family), or a clone derived from a plant or group of plants. A germplasm may be part of an organism or cell, or it may be separate (e.g., isolated) from the organism or cell. In general, germplasm provides genetic material with a specific molecular makeup that is the basis for hereditary qualities of the plant. As used herein, “germplasm” refers to cells of a specific plant; seed; tissue of the specific plant (e.g., tissue from which new plants may be grown); and non-seed parts of the specific plant (e.g., leaf, stem, pollen, and cells). Thus, “germplasm” is used herein synonymously with “genetic material” and may be used to refer to seed (or other plant material) from which a plant may be propagated. A “germplasm bank” may refer to an organized collection of different seed or other genetic material (wherein each genotype is uniquely identified) from which a known cultivar may be cultivated, and from which a new cultivar may be generated. In embodiments, a germplasm utilized in a method or plant as described herein is from a canola line or variety. In particular examples, a germplasm is seed of the canola line or variety. In particular examples, a germplasm is a nucleic acid sample from the Brassica line or variety.

A “haplotype” is the genotype of an individual at a plurality of genetic loci, i.e., a combination of alleles. Typically, the genetic loci described by a haplotype are physically and genetically linked, i.e., on the same chromosome segment.

The terms “increased” or “improved” in connection with “clubroot resistance” is used herein to refer to plants having increased growth, productivity, and/or reduction in root size or number of root nodules, relative a plant that is susceptible (lacking resistance) to clubroot disease, when grown in a field comprising Plasmodiophora brassicae.

The term “introgression” refers to the transmission of an allele at a genetic locus into a genetic background. For example, introgression of a specific allele can involve a sexual cross between two parents of the same species, where at least one of the parents has the specific allele in its genome, to thereby transfer the allele to at least one progeny. Progeny comprising the specific allele form may be repeatedly backcrossed to a line having a desired genetic background. Backcross progeny may be selected for the specific allele form, so as to produce a new variety wherein the specific allele form has been fixed in the progeny's genetic background. In some embodiments, introgression of a specific allele may occur by recombination between two donor genomes (e.g., in a fused protoplast), where at least one of the donor genomes has the specific allele in its genome. Introgression may involve transmission of a specific allele that may be, for example, a selected allele form of a marker allele, a QTL, and/or a transgene.

As used herein an “isolated” biological component (such as a nucleic acid or protein) has been substantially separated, produced apart from, or purified away from other biological components in the cell of the organism in which the component naturally occurs (i.e., other chromosomal and extra-chromosomal DNA and RNA, and proteins), while effecting a chemical or functional change in the component. For example, and without limitation, a nucleic acid may be isolated from a chromosome by breaking chemical bonds connecting the nucleic acid to the remaining DNA in the chromosome and/or the other material previously associated with the nucleic acid in its cellular milieu (e.g., the nucleus). Nucleic acid molecules and proteins that have been “isolated” include nucleic acid molecules and proteins that are enriched or purified. The term also embraces nucleic acids and proteins prepared by recombinant expression in a host cell, as well as chemically-synthesized nucleic acid molecules, proteins, and peptides.

“Marker-assisted selection” (MAS) is a process by which phenotypes are selected based on marker genotypes, i.e., molecular markers. Marker assisted selection can include the use of genetic markers to identify plants for inclusion in and/or removal from a breeding program or planting. A molecular marker allele that demonstrates linkage disequilibrium with a desired phenotypic trait (e.g., a QTL) provides a useful tool for the selection of the desired trait in a plant population. Components for implementing a MAS approach include the creation of a dense (information rich) genetic map of molecular markers in the plant germplasm; the detection of at least one QTL based on statistical associations between marker and phenotypic variability; the definition of a set of particular useful marker alleles based on the results of the QTL analysis; and the use and/or extrapolation of this information to the current set of breeding germplasm to enable marker-based selection decisions to be made.

The closer a particular marker is to a gene that encodes a polypeptide that contributes to a particular phenotype (whether measured in terms of genetic or physical distance), the more tightly linked is the particular marker to the phenotype. In view of the foregoing, it will be appreciated that the closer (whether measured in terms of genetic or physical distance) that a marker is linked to a particular gene or genetic location, the more likely the marker is to segregate with that gene (e.g., a clubroot disease resistance marker disclosed herein) and its associated phenotype (e.g., clubroot disease resistance disclosed herein). Thus, the tightly linked genetic markers for clubroot resistance disclosed herein can be used in MAS programs to identity Brassica varieties that have or can generate progeny that have increased clubroot resistance (relative to parental varieties, breeding population siblings, and/or otherwise isogenic plants lacking that clubroot disease resistance marker), to identify individual plants comprising this clubroot disease resistance trait, and to breed this trait into other Brassica varieties to improve their clubroot disease resistance. Marker-assisted selection is discussed in more detail in a subsection hereinbelow.

A “marker set” or a “set” of markers or probes refers to a specific collection of markers (or data derived therefrom) that may be used to identify individuals comprising a trait of interest. Thus, a set of markers linked to clubroot resistance may be used to identify a Brassica plant comprising one the clubroot disease resistance loci disclosed herein. Data corresponding to a marker set (or data derived from the use of such markers) may be stored in an electronic medium. While each marker in a marker set is useful in the identification of individuals comprising a trait of interest, subsets of markers in a set (i.e., some but not necessarily all of the markers in a marker set) can be used to effectively identify individuals comprising the trait of interest disclosed herein, i.e., one of the clubroot disease resistance loci disclosed herein.

A “modified gene” is a gene that has been mutated or altered through human intervention. Such a “modified” gene has a sequence that differs from the sequence of the corresponding non-modified gene by at least one nucleotide addition, deletion, or substitution. A “modified” plant is a plant comprising a modified gene or deletion.

As used herein the term “native gene” refers to a gene as it is found in its natural endogenous location operably linked to its own regulatory sequences, which have not been altered by human intervention. In the context of this disclosure, a “modified” gene is not a native gene.

As used herein, a ‘nucleic acid molecule” is a polymeric form of nucleotides, which can include both sense and anti-sense strands of RNA, cDNA, genomic DNA, recombinant and synthetic forms and mixed polymers of the above. A nucleotide refers to a ribonucleotide, deoxynucleotide, or a modified form of either type of nucleotide. As used herein “nucleic acid molecule” is synonymous with the terms “nucleic acid”, “nucleotide sequence”, “nucleic acid sequence”, and “polynucleotide.” The term includes single- and double-stranded forms of DNA or RNA. A nucleic acid molecule can refer to either or both naturally occurring and modified nucleotides linked together by naturally occurring and/or non-naturally occurring nucleotide linkages. Nucleic acid molecules may be modified chemically or biochemically, or may contain non-natural or derivatized nucleotide bases, as will be readily appreciated by those of skill in the art. Such modifications include, for example, labels, methylation, substitution of one or more of the naturally occurring nucleotides with an analog, internucleotide modifications, such as uncharged linkages (e.g., methyl phosphonates, phosphotriesters, phosphoramidates, carbamates, etc.), charged linkages (e.g., phosphorothioates, phosphorodithioates, etc.), pendent moieties (e.g., peptides), intercalators (e.g., acridine, psoralen, etc.), chelators, alkylators, and modified linkages (e.g., alpha anomeric nucleic acids, etc.). The term “nucleic acid molecule” also includes any topological conformation, including single-stranded, double stranded, partially duplexed, triplexed, hairpinned, circular, and padlocked conformations. An “endogenous nucleic acid sequence” refers to a nucleic acid sequence within a plant cell, (e.g. an endogenous allele of a native gene present within the genome of a Brassica plant cell).

The term “single-nucleotide polymorphism” (SNP) refers to a DNA sequence variation occurring when a single nucleotide in the genome (or other shared sequence) differs between members of a species or paired chromosomes in an individual. In some examples, markers linked to a clubroot disease resistance locus disclosed herein are SNP markers. Recent high-throughput genotyping technologies such as GoldenGate® and INFINIUM® assays (Illumina, San Diego, CA) may be used in accurate and quick genotyping methods by multiplexing SNPs from 384-plex to >100,000-plex assays per sample.

As used herein, “phenotype” means the detectable characteristics (e.g. clubroot disease resistance) of a cell or organism which can be influenced by genotype.

As used herein, the term “plant material” refers to any processed or unprocessed material derived, in whole or in part, from a plant. For example, and without limitation, a plant material may be a plant part, a seed, a fruit, a leaf, a root, a plant tissue, a plant tissue culture, a plant explant, or a plant cell.

As used herein, the term “plant” may refer to a whole plant, a cell or tissue culture derived from a plant, and/or any part of any of the foregoing. Thus, the term “plant” encompasses, for example and without limitation, whole plants; plant components and/or organs (e.g., leaves, stems, and roots); plant tissue; seed; and a plant cell. A plant cell may be, for example and without limitation, a cell in and/or of a plant, a cell isolated from a plant, and a cell obtained through culturing of a cell isolated from a plant. Thus, the term Brassica “plant” may refer to, for example and without limitation, a whole Brassica plant; multiple Brassica plants; Brassica plant cell(s); Brassica plant protoplast; Brassica tissue culture (e.g., from which a Brassica plant can be regenerated); Brassica plant callus; Brassica plant parts (e.g., seed, flower, cotyledon, leaf, stem, bud, root, and root tip); and Brassica plant cells that are intact in a Brassica plant or in a part of a Brassica plant.

As used herein, a plant or Brassica “line” refers to a group of plants that display little genetic variation (e.g., no genetic variation) between individuals for at least one trait. Inbred lines may be created by several generations of self-pollination and selection or, alternatively, by vegetative propagation from a single parent using tissue or cell culture techniques. As used herein, the terms “cultivar,” “variety,” and “type” are synonymous, and these terms refer to a line that is used for commercial production.

Trait or phenotype: The terms “trait” and “phenotype” are used interchangeably herein. For the purposes of the present disclosure, traits of particular interest are the clubroot disease resistance traits associated with each of the clubroot disease resistance loci disclosed herein.

A “variety” or “cultivar” is a plant line that can be used for commercial production and which is distinct and uniform in its characteristics when propagated. In the case of a hybrid variety or cultivar, the parental lines are distinct, stable, and uniform in their characteristics.

Detection of Disclosed Markers. Each of the markers for the CrM3, CrS3, CrT3, CrB3, and CrN3 loci disclosed herein can be detected by any suitable method for detecting genetic polymorphisms. Suitable methods of detection include nucleotide amplification and/or sequencing of the genomic DNA which will reveal the presence for a disease resistance marker allele disclosed herein for the CrM3, CrS3, CrT3, CrB3, and CrN3 loci. Sec Table 1, Table 2, Table 3, Table 4, and Table 5 (Tables 1-5) disclosing clubroot disease resistance markers alleles for each of the loci disclosed herein.

The clubroot disease resistance marker alleles can be identified and distinguished from susceptible allele using allele-specific amplification and PCR-based amplification assays such as TaqMan, rhAmp-SNP, KASPar, and molecular beacons. Such an assay can include the use of one or more probes that detect the marker allele in (i) nucleic acid that is isolated from a plant or (ii) an amplicon that is selectively amplified by amplification of nucleic acid isolated from a plant. Optionally, such an assay can further include an additional set of primers and/or one or more probes that detect the presence of a clubroot susceptible (e.g., wildtype) allele and thereby determine the zygosity (or even the absence) of clubroot resistance loci disclosed herein.

Additional methods for genotyping and detecting a resistant marker allele for the CrM3, CrS3, CrT3, CrB3, and CrN3 loci disclosed herein (or a linked marker) include but are not limited to, hybridization, primer extension, oligonucleotide ligation, nuclease cleavage, minisequencing and coded spheres. Such methods are reviewed in publications including Gut, 2001, Hum. Mutat. 17:475; Shi, 2001, Clin. Chem. 47:164; Kwok, 2000, Pharmacogenomics 1:95; Bhattramakki and Rafalski, “Discovery and application of single nucleotide polymorphism markers in plants”, in PLANT GENOTYPING: THE DNA FINGERPRINTING OF PLANTS (CABI Publishing, Wallingford 2001). A wide range of commercially available technologies utilize these and other methods to interrogate the allele disclosed herein (or a linked marker), including Masscode™ (Qiagen, Germantown, MD USA), Invader® (Hologic, Madison, WI USA), SnapShot® (Applied Biosystems, Foster City, CA USA.), Taqman® (Applied Biosystems, Foster City, CA USA) and Infinium Bead Chip™ and GoldenGate™ allele-specific extension PCR-based assay (Illumina, San Diego, Calif.).

In particular example, detecting a disclosed maker can include DNA amplification, sequencing, or the combined amplification and sequencing of the marker allele and 5 bp or more, 10 bp or more, 15 bp or more, 20 bp or more, 30 bp or more, 40 bp or more, 50 bp or more, 60 bp or more, 70 bp or more, 80 bp or more, 90 bp or more, 100 bp or more, 110 bp or more, 120 bp or more, 130 bp or more, 140 bp or more, 150 bp or more, 175 bp or more, 200 bp or more, 250 bp or more, 300 bp or more, 350 bp or more, 400 bp or more, 450 bp or more, 500 bp or more, 550 bp or more, or 600 bp or more of flanking sequence that are (i) upstream of (i.e., located 5′ to) the relevant marker allele and/or (ii) downstream of (i.e., located 3′ to) the relevant marker allele. Thus, in particular examples, the disclosed marker can be detected by amplifying genomic sequence to produce an amplicon comprising one or more of the marker allele sequences identified in Tables 1-5 herein. Primers suitable for amplification of each marker are disclosed Tables 1-5. Additionally, the markers disclosed herein can be detected by nucleotide sequencing of genomic DNA (e.g., by first amplifying genomic sequence and sequencing the resulting amplicon) comprising a resistance marker allele sequence identified in Tables 1-5 for each of the disclosed CrM3, CrS3, CrT3, CrB3, and CrN3 loci, respectively.

Other methods of detecting the marker allele for the CrM3, CrS3, CrT3, CrB3, and CrN3 loci disclosed herein include single base extension (SBE) methods, which involve the extension of a nucleotide primer that is adjacent to a polymorphism to incorporate a detectable nucleotide residue upon extension of the primer through the polymorphism, e.g., extension through the marker allele disclosed herein.

Methods of detecting the marker allele for the CrM3, CrS3, CrT3, CrB3, and CrN3 loci disclosed herein also include LCR; and transcription-based amplification methods (e.g., SNP detection, SSR detection, RFLP analysis, and others). Useful techniques include hybridization of a probe nucleic acid to a nucleic acid corresponding to a marker allele disclosed herein, or a linked marker (e.g., an amplified nucleic acid produced using a genomic canola DNA molecule as a template). Hybridization formats including, for example and without limitation, solution phase; solid phase; mixed phase; and in situ hybridization assays may be useful for allele detection in particular embodiments. An extensive guide to hybridization of nucleic acids is discussed in Tijssen, Laboratory Techniques in Biochemistry and Molecular Biology-Hybridization with Nucleic Acid Probes (Elsevier, NY. 1993).

Many detection methods (including amplification-based and sequencing-based methods) may be readily adapted to high throughput analysis in some examples, for example, by using available high throughput sequencing methods, such as sequencing by hybridization.

Detecting each of the CrM3, CrS3, CrT3, CrB3, and CrN3 loci (or marker allele therefor) disclosed herein can be done using nucleotide sequencing products, amplicons, or probes comprising detectable labels. Detectable labels suitable for use include any composition that can be detected by spectroscopic, radioisotopic, photochemical, biochemical, immunochemical, electrical, optical, or chemical means. Thus, a particular allele of a SNP may be detected using, for example, autoradiography, fluorography, or other similar detection techniques, depending on the particular label to be detected. Useful labels include biotin (for staining with labeled streptavidin conjugate), magnetic beads, fluorescent dyes, radiolabels, enzymes, luminescent or phosphorescent indicators, and colorimetric labels. Other labels include ligands that bind to antibodies or specific binding targets labeled with fluorophores, chemiluminescent agents, and enzymes. In some examples the detection techniques disclosed herein include the use of fluorescent dyes (e.g. FAM, VIC, TET, FITC, TRITC, Texas Red, etc.) with or without a quencher (BHQ1 or DABsyl).

Fluorescent dyes useful for labeling probes, primers, and nucleotide 5′-triphosphates include fluoresceins and rhodamines (U.S. Pat. Nos. 5,188,934; 5,366,860; 5,674,442; 5,847,162; 5,885,778; 5,936,087; 6,008,379; 6,020,481; 6,025,505; and 6,051,719), cyanines (Kubista, WO 97/45539) as well as metal porphyrin complexes (Stanton, WO 88/04777). Particular examples include 5-Carboxyfluorescein (5-FAM), 6-carboxyfluorescein (6-FAM), mixtures of 5-FAM ad 6-FAM (5,6-FAM), 2′-chloro-7′phenyl-1,4-dichloro-6-carboxy-fluorescein (VIC), 4,7,2′,4′,5′,7′-hexachloro-6-carboxy-fluorescein (HEX™), 4,7,2′,7′-tetrachloro-6-carboxy-fluorescein (TET), fluorescein isothiocyanate (FITC), 6-carboxy-4′-, 5′-dichloro-2′-, 7′-dimethoxy-fluorescein (JOE), 2′-chloro-5′-fluoro-7′,8′-benzo-1,4-dichloro-6-carboxyfluorescein (NED), 5- and/or 6-carboxytetramethyl-rhodamine (TAMRA), 5- and 6-carboxy-X-rhodamine (ROX), tetramethylrhodamine (TRITC), sulforhodamine 101 acid chloride (Texas Red) and their respective derivatives. Quenchers include nonfluorescent and fluorescent quenchers. Non fluorescent quenchers of particular interest are the diphenyldiazene, more commonly known as azobenzene-derivative class of “dark” quenchers which are commercially available (e.g., from LGC, Biosearch Technologies of Petaluma, CA USA) and include Black Hole Quenchers® or BHQ dyes BHQ-0, BHQ-1, BHQ-2, BHQ-3, etc., as well as DABCYL. Fluorescent quenchers include carboxytetramethyl-rhodamine (TAMRA) and derivatives thereof. Chemiluminescent labels can include 1,2-dioxetane compounds (U.S. Pat. No. 4,931,223; and Bronstein, Anal. Biochemistry 219:169-81 (1994)). Fluorescent dyes, quenchers and chemiluminescent agents are also available from Sigma Aldrich (St. Louis, MO USA).

### Marker Assisted Selection

Molecular markers can be used in a variety of plant breeding applications (e.g. see Staub et al. (1996) Hortscience 31:729-741; Tanksley (1983) Plant Molecular Biology Reporter. 1:3-8). A molecular marker that demonstrates linkage with a locus affecting a desired phenotypic trait provides a useful tool for the selection of the trait in a plant population. This is particularly true where the phenotype is hard to assay. Since DNA marker assays are less laborious and take up less physical space than field phenotyping, much larger populations can be assayed, increasing the chances of finding a recombinant with the target segment from the donor line moved to the recipient line. Thus, marker-assisted selection (MAS) has been used to significantly increase the efficiency of plant breeding at least in part by improving the efficiency of backcrossing and gene introgression.

The closer the linkage between marker and locus, the more useful the marker, as recombination is less likely to occur between the marker and the genomic feature that causes the trait, which can result in false positives. Having flanking markers on both sides of a locus decreases the chances that false positive selection will occur as a double recombination event would be needed. Generally, it is most preferred to have a marker within or at the genomic locus (e.g., within the gene or at the mutation that causes the phenotype) itself, so that recombination cannot occur between the marker and the causal gene or mutation. In some embodiments, the methods disclosed herein produce a marker in a disease resistance gene, wherein the gene was identified by inferring genomic location from clustering of conserved domains or a clustering analysis.

When a gene is introgressed by MAS, it is not only the gene that is introduced but also the flanking regions (Gepts (2002). Crop Sci; 42:1780-1790). This is referred to as “linkage drag.” In the case where the donor plant is highly unrelated to the recipient plant, these flanking regions carry additional genes that may code for agronomically undesirable traits. This “linkage drag” may also result in reduced yield or other negative agronomic characteristics even after multiple cycles of backcrossing into the elite line. This is also sometimes referred to as “yield drag.” The size of the flanking region can be decreased by additional backcrossing, although this is not always successful, as breeders do not have control over the size of the region or the recombination breakpoints (Young et al. (1998) Genetics 120:579-585). In classical breeding it is usually only by chance that recombinations are selected that contribute to a reduction in the size of the donor segment (Tanksley et al. (1989). Biotechnology 7:257-264). Even after 20 backcrosses in backcrosses of this type, one may expect to find a sizeable piece of the donor chromosome still linked to the gene being selected. With markers however, it is possible to select those rare individuals that have experienced recombination near the gene of interest. In 150 backcross plants, there is a 95% chance that at least one plant will have experienced a crossover within 1 cM of the gene, based on a single meiosis map distance. Markers will allow unequivocal identification of those individuals. With one additional backcross of 300 plants, there would be a 95% chance of a crossover within 1 cM single meiosis map distance of the other side of the gene, generating a segment around the target gene of less than 2 cM based on a single meiosis map distance. This can be accomplished in two generations with markers, while it would have required on average 100 generations without markers (See Tanksley et al., supra). When the exact location of a gene is known, flanking markers surrounding the gene can be utilized to select for recombinations in different population sizes. For example, in smaller population sizes, recombinations may be expected further away from the gene, so more distal flanking markers would be required to detect the recombination.

Important components to the implementation of MAS are: (i) defining the population within which the marker-trait association will be determined, which can be a segregating population, or a random or structured population; (ii) monitoring the segregation or association of polymorphic markers relative to the trait, and determining linkage or association using statistical methods; (iii) defining a set of desirable markers based on the results of the statistical analysis, and (iv) the use and/or extrapolation of this information to the current set of breeding germplasm to enable marker-based selection decisions to be made. The markers described in this disclosure, as well as other marker types such as SSRs and FLPs, can be used in marker assisted selection protocols.

SSRs can be defined as relatively short runs of tandemly repeated DNA with lengths of 6 bp or less (Tautz (1989) Nucleic Acid Research 17:6463-6471; Wang et al. (1994) Theoretical and Applied Genetics, 88:1-6) Polymorphisms arise due to variation in the number of repeat units, probably caused by slippage during DNA replication (Levinson and Gutman (1987) Mol Biol Evol 4:203-221). The variation in repeat length may be detected by designing PCR primers to the conserved non-repetitive flanking regions (Weber and May (1989) Am J Hum Genet. 44:388-396). SSRs are highly suited to mapping and MAS as they are multi-allelic, codominant, reproducible, and amenable to high throughput automation (Rafalski et al. (1996) Generating and using DNA markers in plants. In: Non-mammalian genomic analysis: a practical guide. Academic press. pp 75-135).

Various types of SSR markers can be generated, and SSR profiles can be obtained by gel electrophoresis of the amplification products. Scoring of marker genotype is based on the size of the amplified fragment.

Various types of FLP markers can also be generated. Most commonly, amplification primers are used to generate fragment length polymorphisms. Such FLP markers are in many ways similar to SSR markers, except that the region amplified by the primers is not typically a highly repetitive region. Still, the amplified region, or amplicon, will have sufficient variability among germplasm, often due to insertions or deletions, such that the fragments generated by the amplification primers can be distinguished among polymorphic individuals, and such indels are known to occur frequently in maize (Bhattramakki et al. (2002). Plant Mol Biol 48, 539-547; Rafalski (2002b), supra).

SNP markers detect single base pair nucleotide substitutions. Of all the molecular marker types, SNPs are the most abundant, thus having the potential to provide the highest genetic map resolution (Bhattramakki et al. 2002 Plant Molecular Biology 48:539-547). SNPs can be assayed at an even higher level of throughput than SSRs, in a so-called ‘ultra-high-throughput’ fashion, as SNPs do not require large amounts of DNA and automation of the assay may be straight-forward. SNPs also have the promise of being relatively low-cost systems. These three factors together make SNPs highly attractive for use in MAS. Several methods are available for SNP genotyping, including but not limited to, hybridization, primer extension, oligonucleotide ligation, nuclease cleavage, minisequencing, and coded spheres. Such methods have been reviewed in: Gut (2001) Hum Mutat 17 pp. 475-492; Shi (2001) Clin Chem 47, pp. 164-172; Kwok (2000) Pharmacogenomics 1, pp. 95-100; and Bhattramakki and Rafalski (2001) Discovery and application of single nucleotide polymorphism markers in plants. In: R. J. Henry, Ed, Plant Genotyping: The DNA Fingerprinting of Plants, CABI Publishing, Wallingford. A wide range of commercially available technologies utilize these and other methods to interrogate SNPs including Masscode™ (Qiagen), INVADER®. (Third Wave Technologies) and Invader PLUS®, SNAPSHOT®. (Applied Biosystems), TAQMAN®. (Applied Biosystems) and BEADARRAYS®. (Illumina).

A number of SNPs together within a sequence, or across linked sequences, can be used to describe a haplotype for any particular genotype (Ching et al. (2002), BMC Genet. 3:19 pp Gupta et al. 2001, Rafalski (2002b), Plant Science 162:329-333). Haplotypes can be more informative than single SNPs and can be more descriptive of any particular genotype. For example, a single SNP may be allele “T” for a specific line or variety with disease resistance, but the allele ‘T’ might also occur in the breeding population being utilized for recurrent parents. In this case, a haplotype, e.g. a combination of alleles at linked SNP markers, may be more informative. Once a unique haplotype has been assigned to a donor chromosomal region, that haplotype can be used in that population or any subset thereof to determine whether an individual has a particular gene. See, for example, WO2003054229. Using automated high throughput marker detection platforms makes this process highly efficient and effective.

Many of the markers presented herein can readily be used as single nucleotide polymorphic (SNP) markers to select for clubroot resistance. Using PCR, the primers are used to amplify DNA segments from individuals (preferably inbred) that represent the diversity in the population of interest. The PCR products are sequenced directly in one or both directions. The resulting sequences are aligned and polymorphisms are identified. The polymorphisms are not limited to single nucleotide polymorphisms (SNPs), but also include indels, CAPS, SSRs, and VNTRs (variable number of tandem repeats). Specifically, with respect to the fine map information described herein, one can readily use the information provided herein to obtain additional polymorphic SNPs (and other markers) within the region amplified by the primers disclosed herein. Markers within the described map region can be hybridized to BACs or other genomic libraries, or electronically aligned with genome sequences, to find new sequences in the same approximate location as the described markers.

In addition to SSR's, FLPs and SNPs, as described above, other types of molecular markers are also widely used, including but not limited to expressed sequence tags (ESTs), SSR markers derived from EST sequences, randomly amplified polymorphic DNA (RAPD), and other nucleic acid-based markers.

Isozyme profiles and linked morphological characteristics can, in some cases, also be indirectly used as markers. Even though they do not directly detect DNA differences, they are often influenced by specific genetic differences. However, markers that detect DNA variation are far more numerous and polymorphic than isozyme or morphological markers (Tanksley (1983) Plant Molecular Biology Reporter 1:3-8).

Sequence alignments or contigs may also be used to find sequences upstream or downstream of the specific markers listed herein. These new sequences, close to the markers described herein, are then used to discover and develop functionally equivalent markers. For example, different physical and/or genetic maps are aligned to locate equivalent markers not described within this disclosure but that are within similar regions. These maps may be within the species, or even across other species that have been genetically or physically aligned.

In general, MAS uses polymorphic markers that have been identified as having a significant likelihood of co-segregation with a trait such as the clubroot disease resistance traits disclosed herein. Such markers are presumed to map near a gene or genes that give the plant its disease resistant phenotype, and are considered indicators for the desired trait, or markers. Plants are tested for the presence of a desired allele in the marker, and plants containing a desired genotype at one or more loci are expected to transfer the desired genotype, along with a desired phenotype, to their progeny. Thus, plants with clubroot disease resistance may be selected for by detecting one or more marker alleles, and in addition, progeny plants derived from those plants can also be selected. Hence, a plant containing a desired genotype in a given chromosomal region (i.e. a genotype associated with disease resistance) is obtained and then crossed to another plant. The progeny of such a cross would then be evaluated genotypically using one or more markers and the progeny plants with the same genotype in a given chromosomal region would then be selected as having disease resistance.

The markers disclosed herein can be used alone or in combination (i.e. as haplotype) to select for a favorable clubroot resistance locus. For example, each SNP having the resistance allele disclosed in Table 1 (e.g., N88673-001-Q001 having the “T” allele at position 14 of SEQ ID NO: 10) can be used alone or in combination with another SNP resistance allele (e.g., the N88673-001-Q001 having “T” allele and N88667-001-Q001 having the “T” allele at position 11 of SEQ ID NO: 2), or a combination thereof.

The skilled artisan would expect that there might be additional polymorphic sites at marker loci in and around a chromosome marker identified by the methods disclosed herein, wherein one or more polymorphic sites is in linkage disequilibrium (LD) with an allele at one or more of the polymorphic sites in the haplotype and thus could be used in a marker assisted selection program to introgress a gene allele or genomic fragment of interest. Two particular alleles at different polymorphic sites are said to be in LD if the presence of the allele at one of the sites tends to predict the presence of the allele at the other site on the same chromosome (Stevens, Mol. Diag. 4:309-17 (1999)). The marker loci can be located within 5 CM, 2 cM, or 1 cM (on a single meiosis based genetic map) of the disease resistance trait QTL.

The skilled artisan would understand that allelic frequency (and hence, haplotype frequency) can differ from one germplasm pool to another. Germplasm pools vary due to maturity differences, heterotic groupings, geographical distribution, etc. As a result, SNPs and other polymorphisms may not be informative in some germplasm pools.

While the invention has been particularly shown and described with reference to a preferred embodiment and various alternate embodiments, it will be understood by persons skilled in the relevant art that various changes in form and details can be made therein without departing from the spirit and scope of the invention. For instance, while the particular examples below may illustrate the methods and embodiments described herein using a specific plant, the principles in these examples may be applied to any plant. Therefore, it will be appreciated that the scope of this invention is encompassed by the embodiments of the inventions recited herein and in the specification rather than the specific examples that are exemplified below. All cited patents and publications referred to in this application are herein incorporated by reference in their entirety, for all purposes, to the same extent as if each were individually and specifically incorporated by reference.

### EXAMPLES

The following are examples of specific aspects of the invention. The examples are offered for illustrative purposes only, and are not intended to limit the scope of the invention in any way.
Example 1: Screening for Disease Resistance. Corteva Agriscience conducted a large, nearly decade-long research program to identify new, major genetic sources of disease resistance in Brassica. This effort included large-scale genetic screens of Brassica napus (winter oilseed rape and canola), Brassica napus vegetable form (rutabaga) and Brassica rapa (Chinese cabbage and stubble turnip) species which share common genomes. Extensive inter-specific pre-breeding was carried out to introgress resistance gene sources, eliminate linkage drag, characterize their efficacy in different genetic backgrounds, and locate their genomic positions by linkage mapping. One product of this effort was the identification of the genomic hot spots and proprietary markers for clubroot resistance disclosed in the following Examples.
Example 2: Clubroot resistance locus CrM3. Major clubroot resistance locus CrM3 was identified and its genetic position was located to the interval flanked by and including 113.88 cM and 115.57 cM on chromosome N3. One source of this resistance locus has been identified in Mendel, European winter rapeseed (see e.g., Rahman et al., 2011, Canadian Journal of Plant Science, 91 (3), pp. 447-458). The physical position of CrM3 was mapped using proprietary genomic maps to the locus corresponding to nucleotide position 24,092,908 to position 25,040,472 of chromosome N3 of a non-proprietary Brassica napus reference genome. Gene markers were identified within the chromosomal interval and then converted to TaqMan™ (Thermo Fisher, Waltham, MA) assays. CrM3 marker name (NAME), physical position (POS), its resistance allele (RES) and susceptible allele (SUS), SEQ ID NO, and corresponding sequences for assay primers and probes are described in Table 1. These assays were tested on a canola diversity panel comprised of approximately 350 elite lines and hybrids representing the genetic diversity of the proprietary germplasm. The purpose of the canola panel screening was to confirm donor specificity of the markers. TaqMan™ markers were also tested on a proprietary DH mapping population to confirm marker-trait association. None of the markers overlap with publicly available 56k array markers available from Illumina (Madison, WI USA). In Table 1 and in Tables 2-8 herein, the single nucleotide polymorphism SNP for each resistance and susceptibility allele sequence is indicated by bold and underlined text.

TABLE 1

| | | | | SEQ | | |
|---|---|---|---|---|---|---|
| | POS | | | ID | | |
| NAME | (bp) | RES | SUS | NO: | SEQUENCE | FUNCTION |
| N88667 | 23790528 | T | C | 1 | ACTCCGGCACCGAC | FAM Probe for |
| -001- | | | | | | SUS |
| Q001 | | | | 2 | AGGACTCCGGTACCGAC | VIC Probe for |
| | | | | | | RES |
| | | | | 3 | GGAGCCACTACGACAGATAAGAG | Forward |
| | | | | | | Primer |
| | | | | 4 | GTCGGCGCGTGAGC | Reverse Primer |
| N88669 | 24093493 | T | C | 5 | TTGGTGCCGGGATAG | FAM Probe for |
| -001- | | | | | | SUS |
| Q001 | | | | 6 | TTTTGGTGCCAGGATAG | VIC Probe for |
| | | | | | | RES |
| | | | | 7 | ACCTCCGTCGTAAGTAACTTCCTT | Forward |
| | | | | | | Primer |
| | | | | 8 | GGAGAAAAAATTTAGGACGGGTAATAAATGG | Reverse Primer |
| N88673 | 23910470 | T | C | 9 | TTCGAGGAATGTCATGC | FAM Probe for |
| -001- | | | | | | SUS |
| Q001 | | | | 10 | ATTCGAGGAATGTTATGC | VIC Probe for |
| | | | | | | RES |
| | | | | 11 | CATACGGAAGAATTGTTAAAGGGTGAGA | Forward |
| | | | | | | Primer |
| | | | | 12 | AACCCCAATCCCTCACTTATGAAC | Reverse Primer |
| N88677 | 24954854 | A | G | 13 | ACTTTCAGTTCCTGCTCC | FAM Probe for |
| -001- | | | | | | SUS |
| Q001 | | | | 14 | AACTTTCAGTTCTTGCTCC | VIC Probe for |
| | | | | | | RES |
| | | | | 15 | GATCCTCCTAGTTGGCTTTTCCTTT | Forward |
| | | | | | | Primer |
| | | | | 16 | TAAACTCTGAATTTTTAAAACTATTATAAAGT | Reverse Primer |
| | | | | | TGTAAGAC | |
| N88697 | 25172543 | A | C | 17 | CAGAGCACACACAAAA | FAM Probe for |
| -001- | | | | | | SUS |
| Q001 | | | | 18 | CAGAGCACAAACAAAA | VIC Probe for |
| | | | | | | RES |
| | | | | 19 | GAAGTTGTAATGTCATACCCTTCTGTAAAAA | Forward |
| | | | | | AAA | Primer |
| | | | | 20 | ACAGTTGCATATGATGGTAACGATTCA | Reverse Primer |

Each TaqMan™ assay for this Example (as well as the remaining Examples 3-8 herein) was performed using 13.6 μl of a primer probe mixture (18 UM of each probe, 4 UM of each primer) and 1000 μl of master mix from ToughMix™ kit (Quanta Beverly, MA). A liquid handler dispensed 1.3 μl of the mix onto a 1536 well plate containing ˜6 ng of dried DNA. The plate was sealed with a laser sealer and thermocycled in a Hydrocycler device (LGC Genomic Limited, Middlesex, United Kingdom) under the following conditions: 94° C. for 15 min, 40 cycles of 94° C. for 30 secs, 60° C. for 1 min. PCR products are measured using at wavelengths 485 (FAM) and 520 (VIC) by a Pherastar™ plate reader (BMG Labtech, Offenburg, Germany). The values are normalized against ROX and plotted and scored on scatterplots utilizing the Kraken™ software. Marker N88673-001-Q001 was found to be particularly tightly linked to resistance locus CrM3 and was uniquely specific to resistant donor lines.

The clubroot resistance markers in Table 1 are very tightly linked to the CrM3 locus; each has a LOD score of 30 or greater. Furthermore, each of the markers was tested in a mapping population, that included at least 180 individuals. In the test population, each of the marker alleles listed in Table 1 were very tightly linked with the clubroot resistance and clubroot susceptibility traits and all markers were mapped within 2 cM of the CrM3 locus.
Example 3: Clubroot resistance locus CrS3. Another major clubroot resistance locus CrS3 was identified and located to chromosomal interval flanked by and including 113.6 cM and 116.36 cM of chromosome N3. One source of this resistance locus has been identified in Sing, a Chinese cabbage, B. rapa ssp. pekinensis. The physical position of CrS3 was mapped using proprietary genomic maps to the locus corresponding to nucleotide position 23,965,482 to position 24,955,776 of chromosome N3 of a non-proprietary Brassica napus reference genome. Genetic markers located within the chromosomal interval were converted to TaqMan™ assays. Each CrS3 marker (NAME), physical position (POS), its resistance allele (RES) and susceptible allele (SUS), SEQ ID NO, and corresponding sequences for assay primers and probes are described in Table 3. These assays were tested on a canola diversity panel comprised of approximately 350 elite lines and hybrids representing the genetic diversity of the proprietary germplasm and a clubroot donor panel comprised of clubroot resistant donor lines. The purpose of the panel screenings was to confirm donor specificity of the markers. The TaqMan™ markers were also tested on two proprietary DH mapping population to confirm the marker-trait association and four proprietary F2 mapping populations to evaluate the markers' technical performance.

TABLE 2

| | | | | SEQ | | |
|---|---|---|---|---|---|---|
| | POS | | | ID | | |
| NAME | (bp) | RES | SUS | NO: | SEQUENCE | FUNCTION |
| N100CV | 23815398 | T | A | 21 | ACCATGAGACTTTTTAAAA | FAM Probe |
| M-001- | | | | | | for SUS |
| Q001 | | | | 22 | AACCATGAGACTTTTAAAAA | VIC Probe for |
| | | | | | | RES |
| | | | | 23 | TGTCCTAATTTCTACTTGTTTTCTTTTC | Forward |
| | | | | | | Primer |
| | | | | 24 | ATTTATATTAGAAATATTTAATTTAATCT | Reverse |
| | | | | | CTTGCCGACACAC | Primer |
| N100CV | 24092207 | C | T | 25 | AATGACTGAGACAAAAG | FAM Probe |
| N-001- | | | | | | for SUS |
| Q001 | | | | 26 | ATGACTGGGACAAAAG | VIC Probe for |
| | | | | | | RES |
| | | | | 27 | ACCACGTAACATCAATACTATTTTTAATA | Forward |
| | | | | | ATGCTTTTG | Primer |
| | | | | 28 | TTGTGACAGATTGACGCATATT | Reverse |
| | | | | | | Primer |
| N100CV | 24092219 | G | T | 29 | AGCTACCAAATTAAATG | FAM Probe |
| P-001- | | | | | | for SUS |
| Q001 | | | | 30 | AAGCTACCAACTTAAATG | VIC Probe for |
| | | | | | | RES |
| | | | | 31 | CATTGTTTACTTTTGATTGTATAGGCTTTT | Forward |
| | | | | | GT | Primer |
| | | | | 28 | TTGTGACAGATTGACGCATATT | Reverse |
| | | | | | | Primer |
| N100CV | 24170663 | T | C | 32 | CCCGCAAAATCAT | FAM Probe |
| R-001- | | | | | | for SUS |
| Q001 | | | | 33 | ATCCCCGTAAAATCAT | VIC Probe for |
| | | | | | | RES |
| | | | | 34 | CAAGTGAACTTCTTTGGACTTTTTACCA | Forward |
| | | | | | | Primer |
| | | | | 35 | TGGGCCTAGCACCGAATG | Reverse |
| | | | | | | Primer |
| N100CV | 24416406 | T | G | 36 | TGAATGCCGAAGAAA | FAM Probe |
| T-001- | | | | | | for SUS |
| Q001 | | | | 37 | CTGAATGCAGAAGAAA | VIC Probe for |
| | | | | | | RES |
| | | | | 38 | TTTTTTCGTGCACAAGTCAA | Forward |
| | | | | | | Primer |
| | | | | 39 | CGATCCTGAACCAAGTTTGATGAAA | Reverse |
| | | | | | | Primer |
| N100CV | 24651713 | C | T | 40 | TGTTTAAGTACGGAGAGAGA | FAM Probe |
| U-001- | | | | | | for SUS |
| Q001 | | | | 41 | TGTTTAAGTACGGAGAGGGA | VIC Probe for |
| | | | | | | RES |
| | | | | 42 | GTTAGCTTGCTCAGATTAAGTTTTATGAA | Forward |
| | | | | | ATGT | Primer |
| | | | | 43 | GCAAATGGGAACACATATACCATAAAAG | Reverse |
| | | | | | G | Primer |
| N100D0 | 23648444 | T | C | 44 | CTGAAATCACTGGACCTT | FAM Probe |
| A-001- | | | | | | for SUS |
| Q001 | | | | 45 | TTCTGAAATCACTAGACCTT | VIC Probe for |
| | | | | | | RES |
| | | | | 46 | CACCAGCTGGCTAGGCTTA | Forward |
| | | | | | | Primer |
| | | | | 47 | ACGAGTCTGCGATAACGTTCTG | Reverse |
| | | | | | | Primer |
| N100D0 | 24323117 | A | G | 48 | ATTGTTATTGAGTTAGTTTAGTGG | FAM Probe |
| B-001- | | | | | | for SUS |
| Q001 | | | | 49 | TTGTTATTGAGTTAGTTTAATGG | VIC Probe for |
| | | | | | | RES |
| | | | | 50 | GTTGTTTGAGGCCACACTTGAAG | Forward |
| | | | | | | Primer |
| | | | | 51 | GAGAAAATCAATGTTGTGCACTAGGG | Reverse |
| | | | | | | Primer |
| N100D0 | 24447882 | G | T | 52 | ATGGACGTAATATTTCCTC | FAM Probe |
| C-001- | | | | | | for SUS |
| Q001 | | | | 53 | ATGGACGTAATAGTTCCTC | VIC Probe for |
| | | | | | | RES |
| | | | | 54 | ATTTTAAACTCTTATTCAAGCATTTTCAA | Forward |
| | | | | | CTTAACA | Primer |
| | | | | 55 | GTGACCTAAATATATTTCACCAAATTTAA | Reverse |
| | | | | | AATAAATGAATAAGAGTA | Primer |
| N100D0 | 24957105 | A | G | 56 | CTGATTTATCATTGGGCG | FAM Probe |
| D-001- | | | | | | for SUS |
| Q001 | | | | 57 | TCCTGATTTATCATTGGACG | VIC Probe for |
| | | | | | | RES |
| | | | | 58 | ACTTTCCGATTTTGAAGCCCATCTA | Forward |
| | | | | | | Primer |
| | | | | 59 | ACGAACCGAACCATTATTCCAATCA | Reverse |
| | | | | | | Primer |
| N100D0 | :23219681 | A | T | 60 | CACAGTTTGTCACTTAC | FAM Probe |
| M-001- | | | | | | for SUS |
| Q001 | | | | 61 | ACACAGTTTGACACTTAC | VIC Probe for |
| | | | | | | RES |
| | | | | 62 | ATGTTGTGGAGTTATGAAAGGCTCTT | Forward |
| | | | | | | Primer |
| | | | | 63 | TTTGTGGTAAATCAATTCATTTTATGTAG | Reverse |
| | | | | | T | Primer |
| N100D0 | 23221092 | C | T | 64 | CTGATATGGCAACACGTG | FAM Probe |
| N-001- | | | | | | for SUS |
| Q001 | | | | 65 | CTGATATGGCGACACGTG | VIC Probe for |
| | | | | | | RES |
| | | | | 66 | GCTATCCAGCTCAGCAAGGA | Forward |
| | | | | | | Primer |
| | | | | 67 | CAAACAATAGTCTAAAGCCCAATTCGA | Reverse |
| | | | | | | Primer |
| N100D0 | 23236608 | T | C | 68 | CATATCTACTAGTATAGGTATTT | FAM Probe |
| P-001- | | | | | | for SUS |
| Q001 | | | | 69 | ACATATCTACTAGTATAAGTATTT | VIC Probe for |
| | | | | | | RES |
| | | | | 70 | GCAAAACTCTTTTTCGAACAGATACTGAT | Forward |
| | | | | | | Primer |
| | | | | 71 | TCTGAATGTTCATGCAATTTCCAAGT | Reverse |
| | | | | | | Primer |
| N100D0 | 23316014 | C | A | 72 | TGGTTGAATTTGATATAATTTATAG | FAM Probe |
| R-001- | | | | | | for SUS |
| Q001 | | | | 73 | TGGTTGAATTTGATATAATTTAGAG | VIC Probe for |
| | | | | | | RES |
| | | | | 74 | CCAAAATAAACTCGGTTCATTAATCGGTT | Forward |
| | | | | | A | Primer |
| | | | | 75 | CAAATTTTAACCAAATTACACTAGAATCT | Reverse |
| | | | | | CATGGA | Primer |
| N100D0 | 23496732 | T | C | 76 | CGAAGCACATCTCAAAT | FAM Probe |
| T-001- | | | | | | for SUS |
| Q001 | | | | 77 | TCGAAGCACATTTCAAAT | VIC Probe for |
| | | | | | | RES |
| | | | | 78 | CATAAAAACATATCTACTTTTTCTCTCAA | Forward |
| | | | | | ACACAATCT | Primer |
| | | | | 79 | ACCAGTGCCGGAGAAAGAAG | Reverse |
| | | | | | | Primer |
| N100D0 | 23658506 | A | T | 80 | CAATTTAAAACAAAAATATTTTT | FAM Probe |
| U-001- | | | | | | for SUS |
| Q001 | | | | 81 | CAATTTAAAACAAAAATAATTTT | VIC Probe for |
| | | | | | | RES |
| | | | | 82 | GTTTTGTTGTTGATGATATTATATTAACT | Forward |
| | | | | | AGATTTTGTTCCA | Primer |
| | | | | 83 | GAGGTAAATATACATTATTAAACCATAT | Reverse |
| | | | | | AATACATGATCAGAATATTT | Primer |
| N100D0 | 23782716 | T | G | 84 | CGGATACTATTTTCGAGTTTT | FAM Probe |
| V-001- | | | | | | for SUS |
| Q001 | | | | 85 | CGGATACTATTTTCGATTTTT | VIC Probe for |
| | | | | | | RES |
| | | | | 86 | GCTATCTCCGAAATCCAATCTACCA | Forward |
| | | | | | | Primer |
| | | | | 87 | AACGTGGATATGAGATCATGTTTTGGA | Reverse |
| | | | | | | Primer |
| N100D0 | 23802983 | T | A | 88 | TGTAAGTTTCTTAGAGTTCAAG | FAM Probe |
| W-001- | | | | | | for SUS |
| Q001 | | | | 89 | TGTAAGTTTCTTAGAGATCAAG | VIC Probe for |
| | | | | | | RES |
| | | | | 90 | TGAACAGTTACAACCCCATGAAAATATG | Forward |
| | | | | | T | Primer |
| | | | | 91 | CAAAGATTTATTGAATACATCTATATATT | Reverse |
| | | | | | TGTCATGGTAAAATGA | Primer |
| N100D0 | 23809291 | A | G | 92 | CTATATGTATGATATAAAAGCAT | FAM Probe |
| X-001- | | | | | | for SUS |
| Q001 | | | | 93 | TCTATATGTATGATATAAAAACAT | VIC Probe for |
| | | | | | | RES |
| | | | | 94 | CCAAAGGACCTGAGTTGTTAACTCTTA | Forward |
| | | | | | | Primer |
| | | | | 95 | TAGCATGTGAAAATAATTGATAAATGTA | Reverse |
| | | | | | TGAA | Primer |
| N100D0 | 23809456 | A | G | 96 | CTGGAATTGAATAGAATG | FAM Probe |
| Y-001- | | | | | | for SUS |
| Q001 | | | | 97 | ATTTCTGGAATTAAATAGAATG | VIC Probe for |
| | | | | | | RES |
| | | | | 98 | GTGTAACTGGTGACAAATAGAAAGAATA | Forward |
| | | | | | TGTG | Primer |
| | | | | 99 | CAAAAATGTTTTGAAATAAGTGGAATGC | Reverse |
| | | | | | TGATT | Primer |
| N100D1 | 23809466 | A | G | 100 | TGCTGATTCCATTCTAT | FAM Probe |
| 0-001- | | | | | | for SUS |
| Q001 | | | | 101 | AATGCTGATTTCATTCTAT | VIC Probe for |
| | | | | | | RES |
| | | | | 102 | GGTGACAAATAGAAAGAATATGTGGAAT | Forward |
| | | | | | GATAGA | Primer |
| | | | | 103 | ACTTACTGAATGTTTAATTCATGTGCATT | Reverse |
| | | | | | TCAA | Primer |
| N100D1 | 23811780 | C | T | 104 | TGGAAGAAGAAAAGAGACC | FAM Probe |
| 1-001- | | | | | | for SUS |
| Q001 | | | | 105 | ATGGAAGAAGAAGAGAGACC | VIC Probe for |
| | | | | | | RES |
| | | | | 106 | CAGAGCTGCTTTAGCTAGAAAGTCA | Forward |
| | | | | | | Primer |
| | | | | 107 | AGGATATCACAAATTGAAAAATTGTTGA | Reverse |
| | | | | | AAAGGA | Primer |
| N100D1 | 23811916 | A | T | 108 | CGCTCTACTTATTTTTTTAGTTTA | FAM Probe |
| 2-001- | | | | | | for SUS |
| Q001 | | | | 109 | CGCTCTACTTATTTTTTAAGTTTA | VIC Probe for |
| | | | | | | RES |
| | | | | 110 | GCGTATGGTCGGGTATTATTTATGGT | Forward |
| | | | | | | Primer |
| | | | | 111 | CATGAGTACAGCAGCTTTTGAAACTT | Reverse |
| | | | | | | Primer |
| N100D1 | 23816863 | G | T | 112 | CAAAATAGTAGAAAAACTTT | FAM Probe |
| 3-001- | | | | | | for SUS |
| Q001 | | | | 113 | CAAAATAGTAGAACAACTTT | VIC Probe for |
| | | | | | | RES |
| | | | | 114 | GGAATTACTTAGAAGTGGTCATAGCCAA | Forward |
| | | | | | A | Primer |
| | | | | 115 | ACGTACTTGCTAGTTTGAGTTTGGAA | Reverse |
| | | | | | | Primer |
| N100D1 | 23817057 | C | A | 116 | CGTTATAAAAGTCTATTTTTT | FAM Probe |
| 4-001- | | | | | | for SUS |
| Q001 | | | | 117 | ACGTTATAAAAGTCTCTTTTTT | VIC Probe for |
| | | | | | | RES |
| | | | | 118 | CATTTAAAAATGAAATGGTTTACAGTCA | Forward |
| | | | | | GTTTACC | Primer |
| | | | | 119 | AGGACATGAACTGACAACTGTAAGC | Reverse |
| | | | | | | Primer |
| N100D1 | 24090531 | A | T | 120 | CGGTAATCCAGAGTATCA | FAM Probe |
| 5-001- | | | | | | for SUS |
| Q001 | | | | 121 | ATCGGTAATCCAGTGTATCA | VIC Probe for |
| | | | | | | RES |
| | | | | 122 | GTGGATCCTTGACATCCCATGAT | Forward |
| | | | | | | Primer |
| | | | | 123 | TTTCTTTTAGGCATGAGTGTCTGGAT | Reverse |
| | | | | | | Primer |
| N100D1 | 24090662 | A | G | 124 | AATTGGCCACTCCTTT | FAM Probe |
| 6-001- | | | | | | for SUS |
| Q001 | | | | 125 | CAATTGGCCATTCCTTT | VIC Probe for |
| | | | | | | RES |
| | | | | 126 | TCACCTGAGCTTATGCCATATTAAAGAA | Forward |
| | | | | | AA | Primer |
| | | | | 127 | GCTTCATATTTTTGCTTACGAACAAACTT | Reverse |
| | | | | | AGAA | Primer |
| N100D1 | 24090727 | A | C | 128 | TGTTTTGTTTTATTAACTTCT | FAM Probe |
| 7-001- | | | | | | for SUS |
| Q001 | | | | 129 | TTGTTTTGTTTTATTAAATTCT | VIC Probe for |
| | | | | | | RES |
| | | | | 130 | GGCCAATTGGTTTTGGTTAGGTAATT | Forward |
| | | | | | | Primer |
| | | | | 131 | AGTTTGCTTCATATTTTTGCTTACGAACA | Reverse |
| | | | | | | Primer |
| N100D1 | 24090971 | T | A | 132 | TGTTTAAATTTGTATAAATAATTT | FAM Probe |
| 8-001- | | | | | | for SUS |
| Q001 | | | | 133 | ATGTTTAAATTTGTATATATAATTT | VIC Probe for |
| | | | | | | RES |
| | | | | 134 | GTGTGGGATTATTATTAAGTTTAATATAT | Forward |
| | | | | | AATTTGTTTAAGTTTGTGT | Primer |
| | | | | 135 | TATTTTCACATAGTTTGAATATAACACTG | Reverse |
| | | | | | TCAA | Primer |
| N100D1 | 24091058 | A | G | 136 | ACTATGTGAAAATAGTTTATT | FAM Probe |
| 9-001- | | | | | | for SUS |
| Q001 | | | | 137 | AACTATGTGAAAATAATTTATT | VIC Probe for |
| | | | | | | RES |
| | | | | 138 | CACTTTTCCAAGTGTGGGATTATTATTAA | Forward |
| | | | | | GTTTAATAT | Primer |
| | | | | 139 | GTATTAAACCTTGATAGTGACATTTTCT | Reverse |
| | | | | | | Primer |
| N100D1 | 24091376 | C | A | 140 | CCACATTAGTTTAGTTTTAT | FAM Probe |
| A-001- | | | | | | for SUS |
| Q001 | | | | 141 | CCACATTAGTTTCGTTTTAT | VIC Probe for |
| | | | | | | RES |
| | | | | 142 | TGTTTGTAGATAACATTTATTTGTGTGTT | Forward |
| | | | | | TGTTTATCA | Primer |
| | | | | 143 | GTGGGACACAATAAAAATATAAAGTACT | Reverse |
| | | | | | TCATTTCA | Primer |
| N100D1 | 24093151 | A | T | 144 | TCTCCACTTTTATCAAAC | FAM Probe |
| B-001- | | | | | | for SUS |
| Q001 | | | | 145 | TCTCCACTTTAATCAAAC | VIC Probe for |
| | | | | | | RES |
| | | | | 146 | CACGATCACGAGTTGCAAGATG | Forward |
| | | | | | | Primer |
| | | | | 147 | TGAAGGCACCAAAGATATGGAACAG | Reverse |
| | | | | | | Primer |
| N100D1 | 24093258 | T | C | 148 | CAGTTATTCCTCTTACTCG | FAM Probe |
| C-001- | | | | | | for SUS |
| Q001 | | | | 149 | AACAGTTATTCCTTTTACTCG | VIC Probe for |
| | | | | | | RES |
| | | | | 150 | CCCACTTATCAACATCTAA | Forward |
| | | | | | | Primer |
| | | | | 151 | CCCACTTATCAAAATCTAA | Reverse |
| | | | | | | Primer |
| N100D1 | 24093276 | T | C | 152 | TGGTCCGTCGTAAAT | FAM Probe |
| D-001- | | | | | | for SUS |
| Q001 | | | | 153 | ACTGGTCTGTCGTAAAT | VIC Probe for |
| | | | | | | RES |
| | | | | 150 | CCAGCAGAAAGTACTCTAACGATGA | Forward |
| | | | | | | Primer |
| | | | | 151 | TTGATAAGTGGGAATGAGAGGTTACC | Reverse |
| | | | | | | Primer |
| N100D1 | 24093321 | A | C | 154 | CCCACTTATCAACATCTAA | FAM Probe |
| E-001- | | | | | | for SUS |
| Q001 | | | | 155 | CCCACTTATCAAAATCTAA | VIC Probe for |
| | | | | | | RES |
| | | | | 150 | CCCACTTATCAACATCTAA | Forward |
| | | | | | | Primer |
| | | | | 156 | GGAAGCGAGCAGGGAAAGAAA | Reverse |
| | | | | | | Primer |
| N100D1 | 24153890 | T | A | 157 | ATGAAGCAACAATAATGT | FAM Probe |
| F-001- | | | | | | for SUS |
| Q001 | | | | 158 | TGATGAAGCAACATTAATGT | VIC Probe for |
| | | | | | | RES |
| | | | | 159 | GGGAATTTGTTGGATAATCAAAAGGAAA | Forward |
| | | | | | A | Primer |
| | | | | 160 | CAGTTACACTTTGATAATTTTATAATAGG | Reverse |
| | | | | | CAAATTTAGAAAAAAA | Primer |
| N100D1 | 24187359 | G | A | 161 | TCGGTTTAACAATTTCAT | FAM Probe |
| G-001- | | | | | | for SUS |
| Q001 | | | | 162 | CTCGGTTTAACAGTTTCAT | VIC Probe for |
| | | | | | | RES |
| | | | | 163 | TCTTAATTCAAATGAAGTGGTAAGCATTT | Forward |
| | | | | | T | Primer |
| | | | | 164 | CAATGATATGAATTACTGTTAAGGAAAT | Reverse |
| | | | | | ATAATTA | Primer |
| N100D1 | 24414268 | G | A | 165 | TGTGCAATACGTATTTTATA | FAM Probe |
| H-001- | | | | | | for SUS |
| Q001 | | | | 166 | TGTGCAATACGTACTTTATA | VIC Probe for |
| | | | | | | RES |
| | | | | 167 | TTGTGAAAGGGTTTAAACAACTAGAATT | Forward |
| | | | | | TCAT | Primer |
| | | | | 168 | CCGGCCGGTTAACCAATAAAC | Reverse |
| | | | | | | Primer |
| N100D1 | 24439401 | C | T | 169 | CTGAATATTTTCGTGTATTTA | FAM Probe |
| J-001- | | | | | | for SUS |
| Q001 | | | | 170 | ACTGAATATTTTCGTGTGTTTA | VIC Probe for |
| | | | | | | RES |
| | | | | 171 | AGGGTAATTAGAAAAAAGAAAAAGAAA | Forward |
| | | | | | CATGCA | Primer |
| | | | | 172 | CGACATGAGACCTCCAAGTTCA | Reverse |
| | | | | | | Primer |
| N100D1 | 24621733 | A | G | 173 | CATCAGCATTTAGTTTTAT | FAM Probe |
| K-001- | | | | | | for SUS |
| Q001 | | | | 174 | CATCAGCATTTAATTTTAT | VIC Probe for |
| | | | | | | RES |
| | | | | 175 | AACGAGCTCTAGGTTTCAAAACTGT | Forward |
| | | | | | | Primer |
| | | | | 176 | GGTTCCACCAGAAGAAGATCAACAC | Reverse |
| | | | | | | Primer |
| N100D1 | 24655100 | A | G | 177 | TCTTTTGCCAATCTC | FAM Probe |
| M-001- | | | | | | for SUS |
| Q001 | | | | 178 | CTTTCTTTTGTCAATCTC | VIC Probe for |
| | | | | | | RES |
| | | | | 179 | TCAAATCTGGAACCAACGATCGTAA | Forward |
| | | | | | | Primer |
| | | | | 180 | GCCACCAAAACAATAATATAATTCACGT | Reverse |
| | | | | | ATGG | Primer |
| N100D1 | 24943232 | T | A | 181 | CCCGTTTATTTATTTATTTATT | FAM Probe |
| N-001- | | | | | | for SUS |
| Q001 | | | | 182 | ACCCGTTTATTTATTTTTTTATT | VIC Probe for |
| | | | | | | RES |
| | | | | 183 | CAGGAGGCAGTTATTGAGAAAACAGATT | Forward |
| | | | | | AT | Primer |
| | | | | 184 | GGTAAAACAGCGAAAATCCTTATCATGT | Reverse |
| | | | | | | Primer |

Marker N100D1C-001-Q001 was found to be particularly tightly linked to resistance locus CrS3 and was uniquely specific to resistant donor lines.

The clubroot resistance markers in Table 2 are very tightly linked to the CrS3 locus; each has an LOD score of 30 or greater. Furthermore, each of the markers was tested in two different mapping populations. Each test populations included at least 180 individuals. In both test populations, each of the marker alleles listed in Table 2 demonstrated over 90% association with the clubroot resistance and clubroot susceptibility traits.
Example 4: Clubroot resistance locus CrT3. An additional major clubroot resistance locus, CrT3 was identified and its genetic position was located to interval flanked by and including 59.7 cM and 69.8 cM on chromosome N3. One source of this resistance locus has been identified in Tosca, European winter rapeseed (see e.g., Rahman et al., 2011, Canadian Journal of Plant Science, 91 (3), pp. 447-458). The physical position of CrT3 was mapped using proprietary maps to the locus corresponding to nucleotide position 14,777,622 to 16,528,042 of chromosome N3 of a non-proprietary Brassica napus reference genome. Genetic markers linked to CrT3 were converted to TaqMan™ assays. Each CrT3 marker (NAME), physical position (POS), its resistance allele (RES) and susceptible allele (SUS), SEQ ID NO, and corresponding sequences for assay primers and probes are described in Table 3. The assays and tested on a canola diversity panel comprised of approximately 350 elite lines and hybrids representing the genetic diversity of the proprietary germplasm. The purpose of panel screening was to confirm donor specificity of the markers. TaqMan™ markers were also tested on two proprietary DH mapping populations to confirm marker-trait association.

TABLE 3

| | | | | SEQ | | |
|---|---|---|---|---|---|---|
| | POS | | | ID | | |
| NAME | (bp) | RES | SUS | NO: | SEQUENCE | FUNCTION |
| N0014X | 14543573 | G | A | 185 | ATTCAACCAGTTAATAAA | FAM |
| W-001- | | | | | | Probe for |
| Q001 | | | | | | RES |
| | | | | 186 | AAATATTCAACCAATTAATAAA | VIC Probe |
| | | | | | | for SUS |
| | | | | 187 | CAATTGTTCATCAATAATTAGCAGTGGTAAGT | Forward |
| | | | | | | Primer |
| | | | | 188 | GACAATTGCCCTTTACATATCACATGTT | Reverse |
| | | | | | | Primer |
| N0014Y | 14626122 | A | C | 189 | CCTCCTTACAGCCTTG | FAM |
| 6-001- | | | | | | Probe for |
| Q001 | | | | | | SUS |
| | | | | 190 | TTCCTCCTTAAAGCCTTG | VIC Probe |
| | | | | | | for RES |
| | | | | 191 | GTGAGTATTTCAGTGCCACAGTTTT | Forward |
| | | | | | | Primer |
| | | | | 192 | TGACAACAAGTTGAAGGTTTCTCAGA | Reverse |
| | | | | | | Primer |
| N0014Y | 14688286 | G | A | 193 | CGCGCACGTTCCTA | FAM |
| G-001- | | | | | | Probe for |
| Q001 | | | | | | RES |
| | | | | 194 | CGCGCACATTCCTA | VIC Probe |
| | | | | | | for SUS |
| | | | | 195 | CCTGCATACAGACAAGCATTGAATC | Forward |
| | | | | | | Primer |
| | | | | 196 | GCCTCGAGTGTGCTCTTAGC | Reverse |
| | | | | | | Primer |
| N001579 | 14806463 | A | G | 197 | AAACTGATTTACGATTTAG | FAM |
| -001- | | | | | | Probe for |
| Q001 | | | | | | SUS |
| | | | | 198 | AAACTGATTTACAATTTAG | VIC Probe |
| | | | | | | for RES |
| | | | | 199 | CTTCAGGACATTGTATTTACGAAAAAGCA | Forward |
| | | | | | | Primer |
| | | | | 200 | CTTACATGGAATTCCAAGCTGCAAA | Reverse |
| | | | | | | Primer |
| N0015H | 15957400 | A | G | 201 | AACAATCTCTTTGATGACTT | FAM |
| M-001- | | | | | | Probe for |
| Q001 | | | | | | SUS |
| | | | | 202 | AAGAACAATCTCTTTAATGACTT | VIC Probe |
| | | | | | | for RES |
| | | | | 203 | CGTCTCTAGGAACAGCCTTCTTG | Forward |
| | | | | | | Primer |
| | | | | 204 | CCGGTCCTGTGTACAATGTAACTTG | Reverse |
| | | | | | | Primer |
| N0015V | 16244593 | A | C | 205 | CGTCGTCGTGTAGCT | FAM |
| 5-001- | | | | | | Probe for |
| Q001 | | | | | | SUS |
| | | | | 206 | CACGTCGTCTTGTAGCT | VIC Probe |
| | | | | | | for RES |
| | | | | 207 | GGACATATGAACAGTGAATATGCTGAGA | Forward |
| | | | | | | Primer |
| | | | | 208 | GTGTTAAAAAAAGTTTTGAAACATTATCAATT | Reverse |
| | | | | | ATATCAGT | Primer |
| N89533- | 14959178 | T | G | 209 | CGTAAGCATACCTTATTGGTA | FAM |
| 001- | | | | | | Probe for |
| Q001 | | | | | | RES |
| | | | | 210 | ACGTAAGCATACCTTCTTGGTA | VIC Probe |
| | | | | | | for SUS |
| | | | | 211 | AGATAAAAATAAGCATTTATTAAACATTTCAG | Forward |
| | | | | | ACCAAAAATC | Primer |
| | | | | 212 | TGATTATGTAGTCAGGCCGTAATTAAAATTAA | Reverse |
| | | | | | TTGA | Primer |

Markers N89533-001-Q001, N0014XW-001-Q001, N001579-001-Q001, N0015HM-001-Q001, N0014YG-001-Q001, N0014Y6-001-Q001, N0015V5-001-Q001 were found to be particularly tightly linked to resistance locus CrT3 and was uniquely specific for resistant donors.

The clubroot resistance markers in Table 3 are very tightly linked to the CrT3 locus; each has an LOD score of 30 or greater. Furthermore, each of the markers was tested in two different mapping populations. Each test populations included at least 180 individuals. In both test populations, each of the marker alleles listed in Table 3 mapped within the CrT3 QTL interval.
Example 5: Clubroot resistance locus CrB3. One more major clubroot resistance locus CrB3 was identified and its genetic position located to the interval flanked by and including 45.67 cM and 67.79 cM on chromosome N3. One source of this resistance locus has been identified in SW Rebus spring turnip rape from Sweden (see e.g., Tanhuanpaa et al., 2016, Genome 59 (1): 11-21). The physical position of CrB3 was mapped using proprietary maps to the locus corresponding to nucleotide position 10,411,130 to position 15,959,930 of chromosome N3 of a non-proprietary Brassica napus reference genome. Genetic markers located within this chromosomal interval were converted to TaqMan™ assays. Each marker name (NAME), physical position (POS), its resistance allele (RES) and susceptible allele (SUS), SEQ ID NO, and corresponding sequences for assay primers and probes are described in Table 4. The assays were tested on a Brassica napus diversity panel comprised of approximately 350 elite lines and hybrids representing the genetic diversity of the proprietary germplasm and a clubroot donor panel comprising clubroot resistant donor lines. The purpose of the panel screenings was to confirm donor specificity of the markers. TaqMan™ markers were also tested on three proprietary DH mapping population to confirm the marker-trait association.

TABLE 4

| | | | | SEQ | | |
|---|---|---|---|---|---|---|
| | POS | | | ID | | |
| NAME | (bp) | RES | SUS | NO: | SEQUENCE | FUNCTION |
| N23443- | 13331932 | G | C | 213 | TTGTAAATAGGACACATCC | FAM Probe for RES |
| 001- | | | | 214 | TTGTAAATAGGAGACATCC | VIC Probe for SUS |
| Q001 | | | | 215 | GGTTTTCTCTGACAGGTTTTATTTGAA | Forward Primer |
| | | | | | GATATT | |
| | | | | 216 | CACATCAAGGCAAACTTCCAATAGT | Reverse Primer |
| N23448- | 13855648 | G | T | 217 | CACAAGCTCAGTTTC | FAM Probe for SUS |
| 001- | | | | 218 | ACAAGCGCAGTTTC | VIC Probe for RES |
| Q001 | | | | 219 | CATTTTACAGGTAATCAAGCAGAGTCA | Forward Primer |
| | | | | | AG | |
| | | | | 220 | AATTGTCTGCTGCAGGTTTATGC | Reverse Primer |
| N001579 | 14806463 | A | G | 221 | CTGATTTACGATTTAGTACC | FAM Probe for SUS |
| -001- | | | | 222 | CTGATTTACAATTTAGTACCA | VIC Probe for RES |
| Q003 | | | | 223 | CTTCAGGACATTGTATTTACGAAAAAG | Forward Primer |
| | | | | | CA | |
| | | | | 224 | CTTACATGGAATTCCAAGCTGCAAA | Reverse Primer |
| N0015U | 16098951 | A | C | 225 | ATAGTGCATTGATTAAC | FAM Probe for SUS |
| F-001- | | | | 226 | AATTTAATAGTGCATTTATTAAC | VIC Probe for RES |
| Q001 | | | | 227 | CCGTGACAGAGAAATGATGTAGCAA | Forward Primer |
| | | | | 228 | CTACCTTTGAGTTCGTTATTTAACAGC | Reverse Primer |
| | | | | | TT | |
| N0015G | 15595799 | A | C | 229 | AGCCTGAGGTCTAGTTT | FAM Probe for SUS |
| 9-001- | | | | 230 | AGCCTGAGGTATAGTTT | VIC Probe for RES |
| Q001 | | | | 231 | GAATGTTTACTCATAGTTTTTGGCATG | Forward Primer |
| | | | | | GA | |
| | | | | 232 | GGATATTAACCTTACAGTGTCACACAT | Reverse Primer |
| | | | | | ATGC | |
| N0015V | 16244593 | A | C | 233 | CGTCGTCGTGTAGCT | FAM Probe for SUS |
| 5-001- | | | | 234 | CACGTCGTCTTGTAGCT | VIC Probe for RES |
| Q001 | | | | 235 | GGACATATGAACAGTGAATATGCTGA | Forward Primer |
| | | | | | GA | |
| | | | | 236 | GTGTTAAAAAAAGTTTTGAAACATTAT | Reverse Primer |
| | | | | | CAATTATATCAGT | |
| N0014Y | 14752111 | G | A | 237 | CCACCATCAACATC | FAM Probe for RES |
| Y-001- | | | | 238 | TGCCACCATTAACATC | VIC Probe for SUS |
| Q001 | | | | 239 | TGATCAGCTTGCGAAACTTCAAAATAT | Forward Primer |
| | | | | | TG | |
| | | | | 240 | CGACCAGACTCATTTCCTAACCA | Reverse Primer |

Markers N23443-001-Q001, N23448-001-Q001, N001579-001-Q003, N0015UF-001-Q001-Q001, N0015G9-001-Q001, N0015V5-001-Q001, and N0014YY-001-Q001 were found to be particularly tightly linked to resistance locus CrB3. Markers N001579-001-Q003, N0015UF-001-Q001-Q001, N0015G9-001-Q001, N0015V5-001-Q001, and N0014YY-001-Q001 were designed from loci on the publicly available 56k array from Illumina. Markers N23443-001-Q001 and N23448-001-Q001 do not overlap with markers on the Illumina 56k array. While no marker alone is donor specific, these seven markers together created a donor specific rare haplotype. The donor specificity of the haplotype was confirmed using the diversity panel.

The clubroot resistance markers in Table 4 are very tightly linked to the CrB3 locus; each has an LOD score of 30 or greater. Furthermore, each of the markers was tested in three different mapping populations. Each test populations included at least 180 individuals. In all test populations, each of the marker alleles listed in Table 4 demonstrated 100% association with the clubroot resistance and clubroot susceptibility phenotype.
Example 6: Clubroot resistance locus CrN3. Yet another major clubroot resistance locus, CrN3, was identified and its genetic position was located to the interval flanked by and including 62.4 cM and 77.3 cM on chromosome N3. One source of this resistance locus has been identified in Niko, a rutabaga, B. napus ssp. Rapifera, (see e.g., Seguin-Swartz et al., Cruciferae: compendium of trait genetics. Saskatoon Research Centre; 1997). CrN3 was mapped using proprietary maps to the locus corresponding to nucleotide position 14,959,178 to position 17,536,263 of chromosome N3 of a non-proprietary Brassica napus reference genome. Genetic markers located within the chromosomal interval were converted to TaqMan™ assays. Each marker's name (NAME), physical position (POS), its resistance allele (RES) and susceptible allele (SUS), SEQ ID NO, and corresponding sequences for assay primers and probes are described in Table 5. The assays were tested on a Brassica napus (canola/oilseed) diversity panel comprised of approximately 350 elite lines and hybrids representing the genetic diversity of the proprietary germplasm and a clubroot donor panel comprised of clubroot resistant donor lines. The purpose of the panel screenings was to confirm donor specificity of the markers. TaqMan™ markers were also tested on a proprietary DH mapping population to confirm the marker-trait association and a proprietary F2 mapping population to validate the technical performance of the TaqMan™ assays.

TABLE 5

| | | | | SEQ | | |
|---|---|---|---|---|---|---|
| | POS | | | ID | | |
| NAME | (bp) | RES | SUS | NO: | SEQUENCE | FUNCTION |
| N100C | 16656747 | G | T | 241 | TTTCCGGTTTATTAACAAA | FAM Probe for SUS |
| MT- | | | | 242 | TCCGGTTTATGAACAAA | VIC Probe for RES |
| 001- | | | | 243 | GCGGGTTTAGTCATTTTGGTTTTCT | Forward Primer |
| Q001 | | | | 244 | ACGAACGAACCGGATCGG | Reverse Primer |
| N100C | 16054896 | A | G | 245 | AAACTGCTACTTAAAATA | FAM Probe for SUS |
| NM- | | | | 246 | AAATAAAAACTGTTACTTAAAAT | VIC Probe for RES |
| 001- | | | | | A | |
| Q001 | | | | 247 | TTTTTTTGACAATTTGTAAACCAT | Forward Primer |
| | | | | | GC | |
| | | | | 248 | ACAGAAATCAGAGTTATATTATAC | Reverse Primer |
| | | | | | GTTAAATTTATAGATATCAAA | |
| N100C | 16054977 | G | C | 249 | TTGTTATTTTCGAAAAAT | FAM Probe for SUS |
| NN- | | | | 250 | TTTTGTTATTTTCCAAAAAT | VIC Probe for RES |
| 001- | | | | 251 | TTTGATATCTATAAATTTAACGTA | Forward Primer |
| Q001 | | | | | TAATATAACTCTGATTTCTGT | |
| | | | | 252 | TGCTATGTTAGTGTAAGAAACTAT | Reverse Primer |
| | | | | | GAAATATTACGTT | |
| N100C | 16244715 | C | T | 253 | ATGCAACATACAATGTTT | FAM Probe for SUS |
| NP- | | | | 254 | ATGCAACATACGATGTTT | VIC Probe for RES |
| 001- | | | | 255 | CGTGACTGATATAATTGATAATGT | Forward Primer |
| Q001 | | | | | TTCAAAACTTGT | |
| | | | | 256 | ATGTTCATATAATACACACACAAA | Reverse Primer |
| | | | | | CAAGATC | |
| N100C | 16245299 | T | A | 257 | ATGACGATTTGTTTTTGT | FAM Probe for SUS |
| NR- | | | | 258 | ATGACGATTTGATTTTGT | VIC Probe for RES |
| 001- | | | | 259 | TATCTCAAAAAAATTTGTCAACAT | Forward Primer |
| Q001 | | | | | TTTATGTTTTGG | |
| | | | | 260 | TCTCTTTCGATGATTTCTATTTTAG | Reverse Primer |
| | | | | | CATAACGA | |
| N100C | 16245329 | C | G | 261 | CAAACTACCCTTTACTTAGAT | FAM Probe for SUS |
| NT- | | | | 262 | TCAAACTACCCTTTACTTACAT | VIC Probe for RES |
| 001- | | | | 259 | TATCTCAAAAAAATTTGTCAACAT | Forward Primer |
| Q001 | | | | | TTTATGTTTTGG | |
| | | | | 260 | TCTCTTTCGATGATTTCTATTTTAG | Reverse Primer |
| | | | | | CATAACGA | |
| N100C | 16245410 | T | C | 263 | ATGGGAAGCGAATAT | FAM Probe for SUS |
| NU- | | | | 264 | AAGATGGGAAACGAATAT | VIC Probe for RES |
| 001- | | | | 265 | AAATCATCGAAAGAGAAAATTCA | Forward Primer |
| Q001 | | | | | ATTGTCAACT | |
| | | | | 266 | CCAGCAAGCAGTGACGTG | Reverse Primer |
| N100C | 16245699 | A | G | 267 | ATTTTCCCCACAAAAAA | FAM Probe for SUS |
| NV- | | | | 268 | ATTTTTCCCACAAAAAA | VIC Probe for RES |
| 001- | | | | 269 | ACAATGTCCCATTGGTTTATAAAA | Forward Primer |
| Q001 | | | | | TTTACAAAA | |
| | | | | 270 | ACAGAACAACCCCAATACTTTTAA | Reverse Primer |
| | | | | | AGGA | |
| N100C | 16245706 | C | T | 271 | TAAAGGAGCAAATTTT | FAM Probe for SUS |
| NW- | | | | 272 | TAAAGGAGCGAATTTT | VIC Probe for RES |
| 001- | | | | 269 | ACAATGTCCCATTGGTTTATAAAA | Forward Primer |
| Q001 | | | | | TTTACAAAA | |
| | | | | 273 | TTCAAGAAATACAGAACAACCCC | Reverse Primer |
| | | | | | AATACT | |
| N100C | 16795760 | T | A | 274 | ACAACCTTCATCAAAAG | FAM Probe for SUS |
| NX- | | | | 275 | ACAACCTTCAACAAAAG | VIC Probe for RES |
| 001- | | | | 276 | GATGATGATGACGCTGAGGGT | Forward Primer |
| Q001 | | | | 277 | AACTTCTGTAACCACGTGTCTATC | Reverse Primer |
| | | | | | T | |
| N100C | 16445968 | T | C | 278 | CGTCTTCACAAGCCAA | FAM Probe for SUS |
| NY- | | | | 279 | CCCGTCTTCATAAGCCAA | VIC Probe for RES |
| 001- | | | | 280 | GGAGAAAGAGCTTCCGTCACA | Forward Primer |
| Q001 | | | | 281 | CAGGAAGCGTTCGTGTAGCT | Reverse Primer |
| N100C | 16445980 | T | C | 282 | ACGACTCGTGAATTG | FAM Probe for SUS |
| PO-001- | | | | 283 | AGACGACTCATGAATTG | VIC Probe for RES |
| Q001 | | | | 284 | GCTCCAGCTCCCGTCTTC | Forward Primer |
| | | | | 281 | CAGGAAGCGTTCGTGTAGCT | Reverse Primer |
| N100C | 16446193 | C | T | 285 | CTTAAATCACAATCAACTAAT | FAM Probe for SUS |
| P1-001- | | | | 286 | ACTTAAATCACAATCGACTAAT | VIC Probe for RES |
| Q001 | | | | 287 | GCATGCAGGCGTAGGATCT | Forward Primer |
| | | | | 288 | GACTCTTACAAAGACAAGAGCTC | Reverse Primer |
| | | | | | ACTATT | |
| N100C | 16506720 | A | G | 289 | TCTGTCGCGGATGTT | FAM Probe for SUS |
| P2-001- | | | | 290 | TAGGTCTGTCGTGGATGTT | VIC Probe for RES |
| Q001 | | | | 291 | GAAAAAAAGTGAGCAACAAAGGA | Forward Primer |
| | | | | | GGA | |
| | | | | 292 | GTGTTATCTTCATGGTTGTGTTGT | Reverse Primer |
| | | | | | GT | |
| N100C | 16506885 | T | A | 293 | CTTGAACTATCATTAAATAA | FAM Probe for SUS |
| P3-001- | | | | 294 | TCTTGAACTATCAATAAATAA | VIC Probe for RES |
| Q001 | | | | 295 | ACTCGTTCAAACTAAGTAGCAGT | Forward Primer |
| | | | | 296 | CTTGCCTTGAAAGGTATTTGTCTG | Reverse Primer |
| | | | | | TT | |
| N100C | 16302339 | T | A | 297 | ATGGTCTAACTATTTTAATACAAT | FAM Probe for SUS |
| P4-001- | | | | 298 | TGGTCTAACTATTTTAAAACAAT | VIC Probe for RES |
| Q001 | | | | 299 | TAAGGGTGTACTTTTTAATTTACA | Forward Primer |
| | | | | | TTTTTAG | |
| | | | | 300 | TTAAAACAGCAATTTGACATATTC | Reverse Primer |
| | | | | | ATATAA | |
| N100C | 16302411 | T | A | 301 | AGATTATCAAAACAAATGGAT | FAM Probe for SUS |
| P5-001- | | | | 302 | AATAGATTATCAAAACAATTGGA | VIC Probe for RES |
| Q001 | | | | | T | |
| | | | | 303 | TTATATGAATATGTCAAATTGCTG | Forward Primer |
| | | | | | TTTTAA | |
| | | | | 304 | GGTTTAATCATCTACCCATTTCTC | Reverse Primer |
| | | | | | ATCCT | |
| N100C | 16309958 | A | G | 305 | AGCTAAAGATAAAATAAAAATGT | FAM Probe for SUS |
| P6-001- | | | | | A | |
| Q001 | | | | 306 | AGCTAAAGATAAAATAAAAATAT | VIC Probe for RES |
| | | | | | A | |
| | | | | 307 | TCCACTAAATAAATTCTAAAATAA | Forward Primer |
| | | | | | ACACTAATCAT | |
| | | | | 308 | CTATTTAACACTCAAAATAACTGC | Reverse Primer |
| | | | | | AACTTAATATACATCA | |
| N100C | 15881895 | T | G | 309 | AAACCACAGAAAAAT | FAM Probe for SUS |
| P7-001- | | | | 310 | ATGAAACCACATAAAAAT | VIC Probe for RES |
| Q001 | | | | 311 | GCAATGCTTCATTAGCCTATGCTT | Forward Primer |
| | | | | | A | |
| | | | | 312 | CACTAGGCCAACACCTCGTT | Reverse Primer |
| N100C | 16760712 | T | C | 313 | ACGGTGAATAACG | FAM Probe for SUS |
| P8-001- | | | | 314 | TGACGGTAAATAACG | VIC Probe for RES |
| Q001 | | | | 315 | GCATGCATCGTGATTTCTTAAGCA | Forward Primer |
| | | | | | A | |
| | | | | 316 | CCAAACACTCAAATTATAATTTTT | Reverse Primer |
| | | | | | GGTTAATTCCA | |
| N100C | 15542669 | G | A | 317 | TGTGTGGAAACAAAAG | FAM Probe for SUS |
| PA- | | | | 318 | TGTGTGGAAGCAAAAG | VIC Probe for RES |
| 001- | | | | 319 | GATTTTCCAGATAATTGAACGAAC | Forward Primer |
| Q001 | | | | | CTTCTC | |
| | | | | 320 | ACACACACACACATATCTTCCCAA | Reverse Primer |
| | | | | | AA | |
| N100C | 16464221 | T | C | 321 | ACATCTTCAGCTTTTCA | FAM Probe for SUS |
| PB-001- | | | | 322 | AGACATCTTCAGTTTTTCA | VIC Probe for RES |
| Q001 | | | | 323 | GTATTTGCATGTGGTCAGTGTAAC | Forward Primer |
| | | | | | C | |
| | | | | 324 | CAAGGTTGGCAGCAATCATAATTT | Reverse Primer |
| | | | | | CA | |
| N100C | 16884103 | T | C | 325 | CTGATTAATCATGAACTTTTA | FAM Probe for SUS |
| PC-001- | | | | 326 | CTGATTAATCATGAATTTTTA | VIC Probe for RES |
| Q001 | | | | 327 | TCCTAGAATTAATCATGAACTTTT | Forward Primer |
| | | | | | TTTTAACGT | |
| | | | | 328 | GAGGTTGATCTAGATAGAGCAGA | Reverse Primer |
| | | | | | AGA | |
| N100C | 16493696 | C | G | 329 | CAAATGTAGCCCTATTACT | FAM Probe for SUS |
| PD- | | | | 330 | CAAATGTAGCCGTATTACT | VIC Probe for RES |
| 001- | | | | 331 | CTTAAGGTTTTACAAGCTAGACTG | Forward Primer |
| Q001 | | | | | ATTATGACA | |
| | | | | 332 | AGAGATCCAAGCTTGGGAAACAT | Reverse Primer |
| | | | | | AG | |
| N100C | 15831972 | A | G | 333 | CAAATAAAATAACGGCTAAAT | FAM Probe for SUS |
| RA- | | | | 334 | CCAAATAAAATAACGACTAAAT | VIC Probe for RES |
| 001- | | | | 335 | TGCGCGATACTTATGACTGGTTAA | Forward Primer |
| Q001 | | | | | C | |
| | | | | 336 | CGGACTCTATAACCCACTTCTAAA | Reverse Primer |
| | | | | | AACAATA | |
| N100C | 16302333 | G | A | 337 | ATTAATAACTTTTTTTAATAATTG | FAM Probe for SUS |
| RB- | | | | 338 | AATTAATAACTTTTTTTAATGATT | VIC Probe for RES |
| 001- | | | | | G | |
| Q001 | | | | 339 | TAAGGGTGTACTTTTTAATTTACA | Forward Primer |
| | | | | | TTTTTAG | |
| | | | | 340 | TTAAAACAGCAATTTGACATATTC | Reverse Primer |
| | | | | | ATATAA | |
| N100C | 16436970 | A | C | 341 | ATTGGTCAGAAAGAC | FAM Probe for SUS |
| RC- | | | | 342 | ATCTTATTGGTAAGAAAGAC | VIC Probe for RES |
| 001- | | | | 343 | GGGAGGTAAAGTTGAGTTCTTTAT | Forward Primer |
| Q001 | | | | | GGAAT | |
| | | | | 344 | CTACTTATCCACCGGACAAAAGCT | Reverse Primer |
| N100C | 16445768 | T | C | 345 | TGGCTTGAAGACATAGACA | FAM Probe for SUS |
| RD- | | | | 346 | TGGCTTGAAGACATAAACA | VIC Probe for RES |
| 001- | | | | 347 | ATGCCCTCATTTGTAAGATATCAA | Forward Primer |
| Q001 | | | | | GCA | |
| | | | | 348 | GAGAAAATGAGTAAGAAGATCAA | Reverse Primer |
| | | | | | AGCTTCA | |
| N100C | 16464138 | A | G | 349 | AAGAACAAGAAATGGAGTCT | FAM Probe for SUS |
| RE- | | | | 350 | AGAAGAACAAGAAATAGAGTCT | VIC Probe for RES |
| 001- | | | | 351 | CAAGCAGTAGACTCAACAGAATG | Forward Primer |
| Q001 | | | | | C | |
| | | | | 352 | CACTGACCACATGCAAATACTCTA | Reverse Primer |
| | | | | | CATA | |
| N100C | 16506841 | T | C | 353 | CCTGTTGTGATGCATC | FAM Probe for SUS |
| RF-001- | | | | 354 | TGCCTGTTGTAATGCATC | VIC Probe for RES |
| Q001 | | | | 355 | AGCGTAGTTACTTTCCATACTGAA | Forward Primer |
| | | | | | TCAATATAATAG | |
| | | | | 356 | AATGGTTTCCTTTTGATGATTTTTC | Reverse Primer |
| | | | | | TTGAACT | |
| N100C | 16506854 | C | T | 357 | TGAACGAGTACGTGCCTG | FAM Probe for SUS |
| RG- | | | | 358 | AACGAGTGCGTGCCTG | VIC Probe for RES |
| 001- | | | | 359 | CCATACTGAATCAATATAATAGGT | Forward Primer |
| Q001 | | | | | TTTCTATCAAATGC | |
| | | | | 360 | CACCTAGAATAGAATGGTTTCCTT | Reverse Primer |
| | | | | | TTGATG | |
| N100C | 16795812 | A | T | 361 | ATGGTTCTTTATGCTCATAGT | FAM Probe for SUS |
| RJ-001- | | | | 362 | ATGGTTCTTTATGCTCAAAGT | VIC Probe for RES |
| Q001 | | | | 363 | AGGTTGTTCATTTCCTTAATCCAC | Forward Primer |
| | | | | | ACA | |
| | | | | 364 | AACTTCTGTAACCACGTGTCTATC | Reverse Primer |
| | | | | | T | |
| N100C | 16795816 | C | T | 365 | CACATTCAGACAAACT | FAM Probe for SUS |
| RK- | | | | 366 | CTTTCACATTCAGACAGACT | VIC Probe for RES |
| 001- | | | | 363 | AGGTTGTTCATTTCCTTAATCCAC | Forward Primer |
| Q001 | | | | | ACA | |
| | | | | 367 | CCAATCACAGCTCAGTAATGAACC | Reverse Primer |
| | | | | | A | |
| N100C | 15005339 | C | T | 368 | TAGCAAGTTGTTCCTTAATTA | FAM Probe for SUS |
| P9-001- | | | | 369 | AGCAAGTTGTTCCCTAATTA | VIC Probe for RES |
| Q001 | | | | 370 | CTCAATGAATTTACTAAATTACTG | Forward Primer |
| | | | | | TATTTTGTAATCTTCGT | |
| | | | | 371 | CAGACTCTTTTGCCACTTCTCGAT | Reverse Primer |
| | | | | | A | |

Marker N100CP1-001-Q001 was found to be particularly tightly linked to resistance locus CrN3 and was uniquely specific for resistant donor lines.

The clubroot resistance markers in Table 5 are very tightly linked to the CrN3 locus; each has an LOD score of 30 or greater. Furthermore, each of the markers was tested in two different mapping populations. Each test populations included at least 180 individuals. In both test populations, each of the marker alleles listed in Table 5 demonstrated more than 90% association with the clubroot resistance and clubroot susceptibility phenotype.

1-14. (canceled)

15. A method of introducing at least one clubroot resistance locus into a Brassica plant comprising:
crossing a first parent Brassica plant comprising at least one clubroot resistance locus with a second Brassica plant to produce progeny plants;
obtaining a nucleic acid sample from one or more of the progeny plants;
screening each sample for a sequence comprising a molecular marker allele or a haplotype of molecular marker alleles linked to clubroot resistance at one or more of the following loci: CrM3 located on chromosome N3 interval flanked by and including 113.88 cM and 115.57 cM, CrS3 located on chromosome N3 interval flanked by and including 113.6 cM and 116.36 cM, CrT3 located on chromosome N3 interval flanked by and including 59.7 cM and 69.8 cM, CrB3 located on chromosome N3 interval flanked by and including 45.67 cM and 67.79 cM, or CrN3 located on chromosome N3 interval flanked by and including 62.4 cM and 77.3 cM; and
selecting one or more progeny plants comprising the one or more clubroot resistance loci.

16. The method of claim 15, wherein the one or more clubroot resistance loci physical positions on chromosome 3 (Chr 3) correspond to
i) position 24,092,908 to position 25,040,472 of Chr 3;
ii) position 23,965,482 to position 24,955,776 of Chr 3;
iii) position 14,777,622 to position 16,528,042 of Chr 3;
iv) position 10,411,130 to position 15,959,930 of Chr 3; or
v) position 14,959,178 to position 17,536,263 of Chr 3
of reference line DH12075.

17. The method of claim 15, wherein the method further comprises screening the sample for the presence of the molecular marker or haplotype, wherein the molecular marker or haplotype comprises one or more CrM3 resistance alleles identified in Table 1 herein, one or more CrS3 resistance allele identified in Table 2 herein, one or more CrT3 resistance allele identified in Table 3 herein, one or more CrB3 alleles identified in Table 4 herein, or one or more CrN3 resistance alleles identified in Table 5 herein.

18. The method of claim 15, wherein the molecular marker or haplotype comprises one or more of the following alleles:
i) N88673-001-Q001 (SEQ ID NO:10);
ii) N100D1C-001-Q001 (SEQ ID NO:149);
iii) N89533-001-Q001 (SEQ ID NO:209), N0014XW-001-Q001 (SEQ ID NO: 185), N001579-001-Q001 (SEQ ID NO:198), N0015HM-001-Q001 (SEQ ID NO: 202), N0014YG-001-Q001 (SEQ ID NO:193), N0014Y6-001-Q001 (SEQ ID NO: 190), or N0015V5-001-Q001 (SEQ ID NO:206);
iv) N23443-001-Q001 (SEQ ID NO:213), N23448-001-Q001 (SEQ ID NO:218), N001579-001-Q003 (SEQ ID NO:222), N0015UF-001-Q001 (SEQ ID NO:226), N0015G9-001-Q001 (SEQ ID NO:230), N0015V5-001-Q001 (SEQ ID NO:234), N0014YY-001-Q001 (SEQ ID NO:237); or
v) N100CP1-001-Q001 (SEQ ID NO:286).

19. The method of claim 15 further comprising:
crossing the selected one or more progeny plants with the second parent Brassica plant to produce backcross progeny plants.

20. The method of claim 19, further comprising:
obtaining a nucleic acid sample from one or more backcross progeny plants;
screening each sample for a sequence comprising the molecular marker allele or haplotype of molecular marker alleles linked to clubroot resistance at the one or more clubroot resistance loci; and
selecting one or more backcross progeny plants comprising the at least one clubroot resistance loci.

21. The method of claim 20, further comprising:
crossing the selected one or more backcross progeny plants with the second parent Brassica plant to produce additional backcross progeny plants;
screening each sample for a sequence comprising the molecular marker allele or haplotype of molecular marker alleles linked to clubroot resistance at the one or more clubroot resistance loci; and
selecting one or more backcross progeny plants comprising the at least one clubroot resistance loci.

22. The method of claim 21, further comprising repeating the steps of screening and selecting backcross progeny plants two or more additional times to produce further backcross progeny plants that comprise the at least one clubroot resistance locus and the agronomic characteristics of the second parent plant when grown in the same environmental conditions.

23. The method of claim 15, wherein screening each sample comprises the use of a first probe comprising any probe for resistance allele sequence identified in Table 1 as shown in SEQ ID NO: 2, 6, 10, 14, or 18, Table 2 as shown in SEQ ID NO: 22, 26, 30, 33, 37, 41, 45, 49, 53, 57, 61, 65, 69, 73, 77, 81, 85, 89, 93, 97, 101, 105, 109, 113, 117, 121, 125, 129, 133, 137, 141, 145, 149, 153, 155, 158, 162, 166, 170, 174, 178, or 182, Table 3 as shown in SEQ ID NO: 185, 190, 193, 198, 202, 206, 209, Table 4 as shown in SEQ ID NO: 213, 218, 222, 226, 230, 234, or 237, or Table 5 as shown in SEQ ID NO:242, 246, 250, 254, 258, 262, 264, 268, 272, 275, 279, 283, 286, 290, 294, 298, 302, 306, 310, 314, 318, 322, 326, 330, 334, 338, 342, 346, 350, 354, 358, 362, 366, or 369 to thereby detect the presence of a molecular marker allele linked to clubroot resistance.

24. The method of claim 20, wherein screening each sample comprises the use of a first probe comprising any probe for resistance allele sequence identified in Table 1 as shown in SEQ ID NO: 2, 6, 10, 14, or 18, Table 2 as shown in SEQ ID NO: 22, 26, 30, 33, 37, 41, 45, 49, 53, 57, 61, 65, 69, 73, 77, 81, 85, 89, 93, 97, 101, 105, 109, 113, 117, 121, 125, 129, 133, 137, 141, 145, 149, 153, 155, 158, 162, 166, 170, 174, 178, or 182, Table 3 as shown in SEQ ID NO: 185, 190, 193, 198, 202, 206, 209, Table 4 as shown in SEQ ID NO: 213, 218, 222, 226, 230, 234, or 237, or Table 5 as shown in SEQ ID NO:242, 246, 250, 254, 258, 262, 264, 268, 272, 275, 279, 283, 286, 290, 294, 298, 302, 306, 310, 314, 318, 322, 326, 330, 334, 338, 342, 346, 350, 354, 358, 362, 366, or 369 to thereby detect the presence of a molecular marker allele linked to clubroot resistance.

25. A method for determining zygosity of a clubroot resistance allele in a Brassica plant, cell or germplasm thereof, the method comprising:
isolating nucleic acid from a Brassica plant, cell or germplasm thereof;
screening the nucleic acid using a first probe comprising any probe for resistance allele sequence identified in Table 1 as shown in SEQ ID NO: 2, 6, 10, 14, or 18, Table 2 as shown in SEQ ID NO: 22, 26, 30, 33, 37, 41, 45, 49, 53, 57, 61, 65, 69, 73, 77, 81, 85, 89, 93, 97, 101, 105, 109, 113, 117, 121, 125, 129, 133, 137, 141, 145, 149, 153, 155, 158, 162, 166, 170, 174, 178, or 182, Table 3 as shown in SEQ ID NO: 185, 190, 193, 198, 202, 206, 209, Table 4 as shown in SEQ ID NO: 213, 218, 222, 226, 230, 234, or 237, or Table 5 as shown in SEQ ID NO: 242, 246, 250, 254, 258, 262, 264, 268, 272, 275, 279, 283, 286, 290, 294, 298, 302, 306, 310, 314, 318, 322, 326, 330, 334, 338, 342, 346, 350, 354, 358, 362, 366, or 369 and a second probe for susceptibility allele sequence, wherein the first probe is indicative of a marker allele linked to clubroot disease resistance, and the second probe is indicative of a maker allele linked to clubroot disease susceptibility;
quantifying the binding of the first and second probe to the isolated nucleic acid sequence; and,
comparing the quantified binding of the first and second probe to determine zygosity of the clubroot resistance allele.

26. The method of claim 25, wherein the method comprises
amplifying the isolated nucleic acid using a first forward primer comprising a forward primer sequence identified in Table 1, Table 2, Table 3, Table 4, or Table 5 and a first reverse primer comprising a reverse primer sequence identified in Table 1, Table 2, Table 3, Table 4, or Table 5;
screening the amplified nucleic acid using the first probe and the second probe, wherein the second probe is any probe for susceptibility identified in Table 1, Table 2, Table 3, Table 4, or Table 5 herein respectively; and
quantifying the binding of the first and second probe to the amplified nucleic acid sequence.


SEQUENCE LISTING (371 sequences)

SEQ ID NO: 1 - DNA, 14 bp, Artificial Sequence
  synthetic oligonucleotide
actccggcaccgac

SEQ ID NO: 2 - DNA, 17 bp, Artificial Sequence
  synthetic oligonucleotide
aggactccggtaccgac

SEQ ID NO: 3 - DNA, 23 bp, Artificial Sequence
  synthetic oligonucleotide
ggagccactacgacagataagag

SEQ ID NO: 4 - DNA, 14 bp, Artificial Sequence
  synthetic oligonucleotide
gtcggcgcgtgagc

SEQ ID NO: 5 - DNA, 15 bp, Artificial Sequence
  synthetic oligonucleotide
ttggtgccgggatag

SEQ ID NO: 6 - DNA, 17 bp, Artificial Sequence
  synthetic oligonucleotide
ttttggtgccaggatag

SEQ ID NO: 7 - DNA, 24 bp, Artificial Sequence
  synthetic oligonucleotide
acctccgtcgtaagtaacttcctt

SEQ ID NO: 8 - DNA, 31 bp, Artificial Sequence
  synthetic oligonucleotide
ggagaaaaaatttaggacgggtaataaatgg

SEQ ID NO: 9 - DNA, 17 bp, Artificial Sequence
  synthetic oligonucleotide
ttcgaggaatgtcatgc

SEQ ID NO: 10 - DNA, 18 bp, Artificial Sequence
  synthetic oligonucleotide
attcgaggaatgttatgc

SEQ ID NO: 11 - DNA, 28 bp, Artificial Sequence
  synthetic oligonucleotide
catacggaagaattgttaaagggtgaga

SEQ ID NO: 12 - DNA, 24 bp, Artificial Sequence
  synthetic oligonucleotide
aaccccaatccctcacttatgaac

SEQ ID NO: 13 - DNA, 18 bp, Artificial Sequence
  synthetic oligonucleotide
actttcagttcctgctcc

SEQ ID NO: 14 - DNA, 19 bp, Artificial Sequence
  synthetic oligonucleotide
aactttcagttcttgctcc

SEQ ID NO: 15 - DNA, 25 bp, Artificial Sequence
  synthetic oligonucleotide
gatcctcctagttggcttttccttt

SEQ ID NO: 16 - DNA, 40 bp, Artificial Sequence
  synthetic oligonucleotide
taaactctgaatttttaaaactattataaagttgtaagac

SEQ ID NO: 17 - DNA, 16 bp, Artificial Sequence
  synthetic oligonucleotide
cagagcacacacaaaa

SEQ ID NO: 18 - DNA, 16 bp, Artificial Sequence
  synthetic oligonucleotide
cagagcacaaacaaaa

SEQ ID NO: 19 - DNA, 34 bp, Artificial Sequence
  synthetic oligonucleotide
gaagttgtaatgtcatacccttctgtaaaaaaaa

SEQ ID NO: 20 - DNA, 27 bp, Artificial Sequence
  synthetic oligonucleotide
acagttgcatatgatggtaacgattca

SEQ ID NO: 21 - DNA, 19 bp, Artificial Sequence
  synthetic oligonucleotide
accatgagactttttaaaa

SEQ ID NO: 22 - DNA, 20 bp, Artificial Sequence
  synthetic oligonucleotide
aaccatgagacttttaaaaa

SEQ ID NO: 23 - DNA, 28 bp, Artificial Sequence
  synthetic oligonucleotide
tgtcctaatttctacttgttttcttttc

SEQ ID NO: 24 - DNA, 42 bp, Artificial Sequence
  synthetic oligonucleotide
atttatattagaaatatttaatttaatctcttgccgacacac

SEQ ID NO: 25 - DNA, 17 bp, Artificial Sequence
  synthetic oligonucleotide
aatgactgagacaaaag

SEQ ID NO: 26 - DNA, 16 bp, Artificial Sequence
  synthetic oligonucleotide
atgactgggacaaaag

SEQ ID NO: 27 - DNA, 38 bp, Artificial Sequence
  synthetic oligonucleotide
accacgtaacatcaatactatttttaataatgcttttg

SEQ ID NO: 28 - DNA, 22 bp, Artificial Sequence
  synthetic oligonucleotide
ttgtgacagattgacgcatatt

SEQ ID NO: 29 - DNA, 17 bp, Artificial Sequence
  synthetic oligonucleotide
agctaccaaattaaatg

SEQ ID NO: 30 - DNA, 18 bp, Artificial Sequence
  synthetic oligonucleotide
aagctaccaacttaaatg

SEQ ID NO: 31 - DNA, 32 bp, Artificial Sequence
  synthetic oligonucleotide
cattgtttacttttgattgtataggcttttgt

SEQ ID NO: 32 - DNA, 13 bp, Artificial Sequence
  synthetic oligonucleotide
cccgcaaaatcat

SEQ ID NO: 33 - DNA, 16 bp, Artificial Sequence
  synthetic oligonucleotide
atccccgtaaaatcat

SEQ ID NO: 34 - DNA, 28 bp, Artificial Sequence
  synthetic oligonucleotide
caagtgaacttctttggactttttacca

SEQ ID NO: 35 - DNA, 18 bp, Artificial Sequence
  synthetic oligonucleotide
tgggcctagcaccgaatg

SEQ ID NO: 36 - DNA, 15 bp, Artificial Sequence
  synthetic oligonucleotide
tgaatgccgaagaaa

SEQ ID NO: 37 - DNA, 16 bp, Artificial Sequence
  synthetic oligonucleotide
ctgaatgcagaagaaa

SEQ ID NO: 38 - DNA, 20 bp, Artificial Sequence
  synthetic oligonucleotide
ttttttcgtgcacaagtcaa

SEQ ID NO: 39 - DNA, 25 bp, Artificial Sequence
  synthetic oligonucleotide
cgatcctgaaccaagtttgatgaaa

SEQ ID NO: 40 - DNA, 20 bp, Artificial Sequence
  synthetic oligonucleotide
tgtttaagtacggagagaga

SEQ ID NO: 41 - DNA, 20 bp, Artificial Sequence
  synthetic oligonucleotide
tgtttaagtacggagaggga

SEQ ID NO: 42 - DNA, 33 bp, Artificial Sequence
  synthetic oligonucleotide
gttagcttgctcagattaagttttatgaaatgt

SEQ ID NO: 43 - DNA, 29 bp, Artificial Sequence
  synthetic oligonucleotide
gcaaatgggaacacatataccataaaagg

SEQ ID NO: 44 - DNA, 18 bp, Artificial Sequence
  synthetic oligonucleotide
ctgaaatcactggacctt

SEQ ID NO: 45 - DNA, 20 bp, Artificial Sequence
  synthetic oligonucleotide
ttctgaaatcactagacctt

SEQ ID NO: 46 - DNA, 19 bp, Artificial Sequence
  synthetic oligonucleotide
caccagctggctaggctta

SEQ ID NO: 47 - DNA, 22 bp, Artificial Sequence
  synthetic oligonucleotide
acgagtctgcgataacgttctg

SEQ ID NO: 48 - DNA, 24 bp, Artificial Sequence
  synthetic oligonucleotide
attgttattgagttagtttagtgg

SEQ ID NO: 49 - DNA, 23 bp, Artificial Sequence
  synthetic oligonucleotide
ttgttattgagttagtttaatgg

SEQ ID NO: 50 - DNA, 23 bp, Artificial Sequence
  synthetic oligonucleotide
gttgtttgaggccacacttgaag

SEQ ID NO: 51 - DNA, 26 bp, Artificial Sequence
  synthetic oligonucleotide
gagaaaatcaatgttgtgcactaggg

SEQ ID NO: 52 - DNA, 19 bp, Artificial Sequence
  synthetic oligonucleotide
atggacgtaatatttcctc

SEQ ID NO: 53 - DNA, 19 bp, Artificial Sequence
  synthetic oligonucleotide
atggacgtaatagttcctc

SEQ ID NO: 54 - DNA, 36 bp, Artificial Sequence
  synthetic oligonucleotide
attttaaactcttattcaagcattttcaacttaaca

SEQ ID NO: 55 - DNA, 47 bp, Artificial Sequence
  synthetic oligonucleotide
gtgacctaaatatatttcaccaaatttaaaataaatgaataagagta

SEQ ID NO: 56 - DNA, 18 bp, Artificial Sequence
  synthetic oligonucleotide
ctgatttatcattgggcg

SEQ ID NO: 57 - DNA, 20 bp, Artificial Sequence
  synthetic oligonucleotide
tcctgatttatcattggacg

SEQ ID NO: 58 - DNA, 25 bp, Artificial Sequence
  synthetic oligonucleotide
actttccgattttgaagcccatcta

SEQ ID NO: 59 - DNA, 25 bp, Artificial Sequence
  synthetic oligonucleotide
acgaaccgaaccattattccaatca

SEQ ID NO: 60 - DNA, 17 bp, Artificial Sequence
  synthetic oligonucleotide
cacagtttgtcacttac

SEQ ID NO: 61 - DNA, 18 bp, Artificial Sequence
  synthetic oligonucleotide
acacagtttgacacttac

SEQ ID NO: 62 - DNA, 26 bp, Artificial Sequence
  synthetic oligonucleotide
atgttgtggagttatgaaaggctctt

SEQ ID NO: 63 - DNA, 30 bp, Artificial Sequence
  synthetic oligonucleotide
tttgtggtaaatcaattcattttatgtagt

SEQ ID NO: 64 - DNA, 18 bp, Artificial Sequence
  synthetic oligonucleotide
ctgatatggcaacacgtg

SEQ ID NO: 65 - DNA, 18 bp, Artificial Sequence
  synthetic oligonucleotide
ctgatatggcgacacgtg

SEQ ID NO: 66 - DNA, 20 bp, Artificial Sequence
  synthetic oligonucleotide
gctatccagctcagcaagga

SEQ ID NO: 67 - DNA, 27 bp, Artificial Sequence
  synthetic oligonucleotide
caaacaatagtctaaagcccaattcga

SEQ ID NO: 68 - DNA, 23 bp, Artificial Sequence
  synthetic oligonucleotide
catatctactagtataggtattt

SEQ ID NO: 69 - DNA, 24 bp, Artificial Sequence
  synthetic oligonucleotide
acatatctactagtataagtattt

SEQ ID NO: 70 - DNA, 29 bp, Artificial Sequence
  synthetic oligonucleotide
gcaaaactctttttcgaacagatactgat

SEQ ID NO: 71 - DNA, 26 bp, Artificial Sequence
  synthetic oligonucleotide
tctgaatgttcatgcaatttccaagt

SEQ ID NO: 72 - DNA, 25 bp, Artificial Sequence
  synthetic oligonucleotide
tggttgaatttgatataatttatag

SEQ ID NO: 73 - DNA, 25 bp, Artificial Sequence
  synthetic oligonucleotide
tggttgaatttgatataatttagag

SEQ ID NO: 74 - DNA, 30 bp, Artificial Sequence
  synthetic oligonucleotide
ccaaaataaactcggttcattaatcggtta

SEQ ID NO: 75 - DNA, 35 bp, Artificial Sequence
  synthetic oligonucleotide
caaattttaaccaaattacactagaatctcatgga

SEQ ID NO: 76 - DNA, 17 bp, Artificial Sequence
  synthetic oligonucleotide
cgaagcacatctcaaat

SEQ ID NO: 77 - DNA, 18 bp, Artificial Sequence
  synthetic oligonucleotide
tcgaagcacatttcaaat

SEQ ID NO: 78 - DNA, 38 bp, Artificial Sequence
  synthetic oligonucleotide
cataaaaacatatctactttttctctcaaacacaatct

SEQ ID NO: 79 - DNA, 20 bp, Artificial Sequence
  synthetic oligonucleotide
accagtgccggagaaagaag

SEQ ID NO: 80 - DNA, 23 bp, Artificial Sequence
  synthetic oligonucleotide
caatttaaaacaaaaatattttt

SEQ ID NO: 81 - DNA, 23 bp, Artificial Sequence
  synthetic oligonucleotide
caatttaaaacaaaaataatttt

SEQ ID NO: 82 - DNA, 42 bp, Artificial Sequence
  synthetic oligonucleotide
gttttgttgttgatgatattatattaactagattttgttcca

SEQ ID NO: 83 - DNA, 48 bp, Artificial Sequence
  synthetic oligonucleotide
gaggtaaatatacattattaaaccatataatacatgatcagaatattt

SEQ ID NO: 84 - DNA, 21 bp, Artificial Sequence
  synthetic oligonucleotide
cggatactattttcgagtttt

SEQ ID NO: 85 - DNA, 21 bp, Artificial Sequence
  synthetic oligonucleotide
cggatactattttcgattttt

SEQ ID NO: 86 - DNA, 25 bp, Artificial Sequence
  synthetic oligonucleotide
gctatctccgaaatccaatctacca

SEQ ID NO: 87 - DNA, 27 bp, Artificial Sequence
  synthetic oligonucleotide
aacgtggatatgagatcatgttttgga

SEQ ID NO: 88 - DNA, 22 bp, Artificial Sequence
  synthetic oligonucleotide
tgtaagtttcttagagttcaag

SEQ ID NO: 89 - DNA, 22 bp, Artificial Sequence
  synthetic oligonucleotide
tgtaagtttcttagagatcaag

SEQ ID NO: 90 - DNA, 29 bp, Artificial Sequence
  synthetic oligonucleotide
tgaacagttacaaccccatgaaaatatgt

SEQ ID NO: 91 - DNA, 45 bp, Artificial Sequence
  synthetic oligonucleotide
caaagatttattgaatacatctatatatttgtcatggtaaaatga

SEQ ID NO: 92 - DNA, 23 bp, Artificial Sequence
  synthetic oligonucleotide
ctatatgtatgatataaaagcat

SEQ ID NO: 93 - DNA, 24 bp, Artificial Sequence
  synthetic oligonucleotide
tctatatgtatgatataaaaacat

SEQ ID NO: 94 - DNA, 27 bp, Artificial Sequence
  synthetic oligonucleotide
ccaaaggacctgagttgttaactctta

SEQ ID NO: 95 - DNA, 32 bp, Artificial Sequence
  synthetic oligonucleotide
tagcatgtgaaaataattgataaatgtatgaa

SEQ ID NO: 96 - DNA, 18 bp, Artificial Sequence
  synthetic oligonucleotide
ctggaattgaatagaatg

SEQ ID NO: 97 - DNA, 22 bp, Artificial Sequence
  synthetic oligonucleotide
atttctggaattaaatagaatg

SEQ ID NO: 98 - DNA, 32 bp, Artificial Sequence
  synthetic oligonucleotide
gtgtaactggtgacaaatagaaagaatatgtg

SEQ ID NO: 99 - DNA, 33 bp, Artificial Sequence
  synthetic oligonucleotide
caaaaatgttttgaaataagtggaatgctgatt

SEQ ID NO: 100 - DNA, 17 bp, Artificial Sequence
  synthetic oligonucleotide
tgctgattccattctat

SEQ ID NO: 101 - DNA, 19 bp, Artificial Sequence
  synthetic oligonucleotide
aatgctgatttcattctat

SEQ ID NO: 102 - DNA, 34 bp, Artificial Sequence
  synthetic oligonucleotide
ggtgacaaatagaaagaatatgtggaatgataga

SEQ ID NO: 103 - DNA, 33 bp, Artificial Sequence
  synthetic oligonucleotide
acttactgaatgtttaattcatgtgcatttcaa

SEQ ID NO: 104 - DNA, 19 bp, Artificial Sequence
  synthetic oligonucleotide
tggaagaagaaaagagacc

SEQ ID NO: 105 - DNA, 20 bp, Artificial Sequence
  synthetic oligonucleotide
atggaagaagaagagagacc

SEQ ID NO: 106 - DNA, 25 bp, Artificial Sequence
  synthetic oligonucleotide
cagagctgctttagctagaaagtca

SEQ ID NO: 107 - DNA, 34 bp, Artificial Sequence
  synthetic oligonucleotide
aggatatcacaaattgaaaaattgttgaaaagga

SEQ ID NO: 108 - DNA, 24 bp, Artificial Sequence
  synthetic oligonucleotide
cgctctacttatttttttagttta

SEQ ID NO: 109 - DNA, 24 bp, Artificial Sequence
  synthetic oligonucleotide
cgctctacttattttttaagttta

SEQ ID NO: 110 - DNA, 26 bp, Artificial Sequence
  synthetic oligonucleotide
gcgtatggtcgggtattatttatggt

SEQ ID NO: 111 - DNA, 26 bp, Artificial Sequence
  synthetic oligonucleotide
catgagtacagcagcttttgaaactt

SEQ ID NO: 112 - DNA, 20 bp, Artificial Sequence
  synthetic oligonucleotide
caaaatagtagaaaaacttt

SEQ ID NO: 113 - DNA, 20 bp, Artificial Sequence
  synthetic oligonucleotide
caaaatagtagaacaacttt

SEQ ID NO: 114 - DNA, 29 bp, Artificial Sequence
  synthetic oligonucleotide
ggaattacttagaagtggtcatagccaaa

SEQ ID NO: 115 - DNA, 26 bp, Artificial Sequence
  synthetic oligonucleotide
acgtacttgctagtttgagtttggaa

SEQ ID NO: 116 - DNA, 21 bp, Artificial Sequence
  synthetic oligonucleotide
cgttataaaagtctatttttt

SEQ ID NO: 117 - DNA, 22 bp, Artificial Sequence
  synthetic oligonucleotide
acgttataaaagtctctttttt

SEQ ID NO: 118 - DNA, 35 bp, Artificial Sequence
  synthetic oligonucleotide
catttaaaaatgaaatggtttacagtcagtttacc

SEQ ID NO: 119 - DNA, 25 bp, Artificial Sequence
  synthetic oligonucleotide
aggacatgaactgacaactgtaagc

SEQ ID NO: 120 - DNA, 18 bp, Artificial Sequence
  synthetic oligonucleotide
cggtaatccagagtatca

SEQ ID NO: 121 - DNA, 20 bp, Artificial Sequence
  synthetic oligonucleotide
atcggtaatccagtgtatca

SEQ ID NO: 122 - DNA, 23 bp, Artificial Sequence
  synthetic oligonucleotide
gtggatccttgacatcccatgat

SEQ ID NO: 123 - DNA, 26 bp, Artificial Sequence
  synthetic oligonucleotide
tttcttttaggcatgagtgtctggat

SEQ ID NO: 124 - DNA, 16 bp, Artificial Sequence
  synthetic oligonucleotide
aattggccactccttt

SEQ ID NO: 125 - DNA, 17 bp, Artificial Sequence
  synthetic oligonucleotide
caattggccattccttt

SEQ ID NO: 126 - DNA, 30 bp, Artificial Sequence
  synthetic oligonucleotide
tcacctgagcttatgccatattaaagaaaa

SEQ ID NO: 127 - DNA, 33 bp, Artificial Sequence
  synthetic oligonucleotide
gcttcatatttttgcttacgaacaaacttagaa

SEQ ID NO: 128 - DNA, 21 bp, Artificial Sequence
  synthetic oligonucleotide
tgttttgttttattaacttct

SEQ ID NO: 129 - DNA, 22 bp, Artificial Sequence
  synthetic oligonucleotide
ttgttttgttttattaaattct

SEQ ID NO: 130 - DNA, 26 bp, Artificial Sequence
  synthetic oligonucleotide
ggccaattggttttggttaggtaatt

SEQ ID NO: 131 - DNA, 29 bp, Artificial Sequence
  synthetic oligonucleotide
agtttgcttcatatttttgcttacgaaca

SEQ ID NO: 132 - DNA, 24 bp, Artificial Sequence
  synthetic oligonucleotide
tgtttaaatttgtataaataattt

SEQ ID NO: 133 - DNA, 25 bp, Artificial Sequence
  synthetic oligonucleotide
atgtttaaatttgtatatataattt

SEQ ID NO: 134 - DNA, 48 bp, Artificial Sequence
  synthetic oligonucleotide
gtgtgggattattattaagtttaatatataatttgtttaagtttgtgt

SEQ ID NO: 135 - DNA, 33 bp, Artificial Sequence
  synthetic oligonucleotide
tattttcacatagtttgaatataacactgtcaa

SEQ ID NO: 136 - DNA, 21 bp, Artificial Sequence
  synthetic oligonucleotide
actatgtgaaaatagtttatt

SEQ ID NO: 137 - DNA, 22 bp, Artificial Sequence
  synthetic oligonucleotide
aactatgtgaaaataatttatt

SEQ ID NO: 138 - DNA, 38 bp, Artificial Sequence
  synthetic oligonucleotide
cacttttccaagtgtgggattattattaagtttaatat

SEQ ID NO: 139 - DNA, 28 bp, Artificial Sequence
  synthetic oligonucleotide
gtattaaaccttgatagtgacattttct

SEQ ID NO: 140 - DNA, 20 bp, Artificial Sequence
  synthetic oligonucleotide
ccacattagtttagttttat

SEQ ID NO: 141 - DNA, 20 bp, Artificial Sequence
  synthetic oligonucleotide
ccacattagtttcgttttat

SEQ ID NO: 142 - DNA, 38 bp, Artificial Sequence
  synthetic oligonucleotide
tgtttgtagataacatttatttgtgtgtttgtttatca

SEQ ID NO: 143 - DNA, 36 bp, Artificial Sequence
  synthetic oligonucleotide
gtgggacacaataaaaatataaagtacttcatttca

SEQ ID NO: 144 - DNA, 18 bp, Artificial Sequence
  synthetic oligonucleotide
tctccacttttatcaaac

SEQ ID NO: 145 - DNA, 18 bp, Artificial Sequence
  synthetic oligonucleotide
tctccactttaatcaaac

SEQ ID NO: 146 - DNA, 22 bp, Artificial Sequence
  synthetic oligonucleotide
cacgatcacgagttgcaagatg

SEQ ID NO: 147 - DNA, 25 bp, Artificial Sequence
  synthetic oligonucleotide
tgaaggcaccaaagatatggaacag

SEQ ID NO: 148 - DNA, 19 bp, Artificial Sequence
  synthetic oligonucleotide
cagttattcctcttactcg

SEQ ID NO: 149 - DNA, 21 bp, Artificial Sequence
  synthetic oligonucleotide
aacagttattccttttactcg

SEQ ID NO: 150 - DNA, 25 bp, Artificial Sequence
  synthetic oligonucleotide
ccagcagaaagtactctaacgatga

SEQ ID NO: 151 - DNA, 26 bp, Artificial Sequence
  synthetic oligonucleotide
ttgataagtgggaatgagaggttacc

SEQ ID NO: 152 - DNA, 15 bp, Artificial Sequence
  synthetic oligonucleotide
tggtccgtcgtaaat

SEQ ID NO: 153 - DNA, 17 bp, Artificial Sequence
  synthetic oligonucleotide
actggtctgtcgtaaat

SEQ ID NO: 154 - DNA, 19 bp, Artificial Sequence
  synthetic oligonucleotide
cccacttatcaacatctaa

SEQ ID NO: 155 - DNA, 19 bp, Artificial Sequence
  synthetic oligonucleotide
cccacttatcaaaatctaa

SEQ ID NO: 156 - DNA, 21 bp, Artificial Sequence
  synthetic oligonucleotide
ggaagcgagcagggaaagaaa

SEQ ID NO: 157 - DNA, 18 bp, Artificial Sequence
  synthetic oligonucleotide
atgaagcaacaataatgt

SEQ ID NO: 158 - DNA, 20 bp, Artificial Sequence
  synthetic oligonucleotide
tgatgaagcaacattaatgt

SEQ ID NO: 159 - DNA, 29 bp, Artificial Sequence
  synthetic oligonucleotide
gggaatttgttggataatcaaaaggaaaa

SEQ ID NO: 160 - DNA, 45 bp, Artificial Sequence
  synthetic oligonucleotide
cagttacactttgataattttataataggcaaatttagaaaaaaa

SEQ ID NO: 161 - DNA, 18 bp, Artificial Sequence
  synthetic oligonucleotide
tcggtttaacaatttcat

SEQ ID NO: 162 - DNA, 19 bp, Artificial Sequence
  synthetic oligonucleotide
ctcggtttaacagtttcat

SEQ ID NO: 163 - DNA, 30 bp, Artificial Sequence
  synthetic oligonucleotide
tcttaattcaaatgaagtggtaagcatttt

SEQ ID NO: 164 - DNA, 35 bp, Artificial Sequence
  synthetic oligonucleotide
caatgatatgaattactgttaaggaaatataatta

SEQ ID NO: 165 - DNA, 20 bp, Artificial Sequence
  synthetic oligonucleotide
tgtgcaatacgtattttata

SEQ ID NO: 166 - DNA, 20 bp, Artificial Sequence
  synthetic oligonucleotide
tgtgcaatacgtactttata

SEQ ID NO: 167 - DNA, 32 bp, Artificial Sequence
  synthetic oligonucleotide
ttgtgaaagggtttaaacaactagaatttcat

SEQ ID NO: 168 - DNA, 21 bp, Artificial Sequence
  synthetic oligonucleotide
ccggccggttaaccaataaac

SEQ ID NO: 169 - DNA, 21 bp, Artificial Sequence
  synthetic oligonucleotide
ctgaatattttcgtgtattta

SEQ ID NO: 170 - DNA, 22 bp, Artificial Sequence
  synthetic oligonucleotide
actgaatattttcgtgtgttta

SEQ ID NO: 171 - DNA, 33 bp, Artificial Sequence
  synthetic oligonucleotide
agggtaattagaaaaaagaaaaagaaacatgca

SEQ ID NO: 172 - DNA, 22 bp, Artificial Sequence
  synthetic oligonucleotide
cgacatgagacctccaagttca

SEQ ID NO: 173 - DNA, 19 bp, Artificial Sequence
  synthetic oligonucleotide
catcagcatttagttttat

SEQ ID NO: 174 - DNA, 19 bp, Artificial Sequence
  synthetic oligonucleotide
catcagcatttaattttat

SEQ ID NO: 175 - DNA, 25 bp, Artificial Sequence
  synthetic oligonucleotide
aacgagctctaggtttcaaaactgt

SEQ ID NO: 176 - DNA, 25 bp, Artificial Sequence
  synthetic oligonucleotide
ggttccaccagaagaagatcaacac

SEQ ID NO: 177 - DNA, 15 bp, Artificial Sequence
  synthetic oligonucleotide
tcttttgccaatctc

SEQ ID NO: 178 - DNA, 18 bp, Artificial Sequence
  synthetic oligonucleotide
ctttcttttgtcaatctc

SEQ ID NO: 179 - DNA, 25 bp, Artificial Sequence
  synthetic oligonucleotide
tcaaatctggaaccaacgatcgtaa

SEQ ID NO: 180 - DNA, 32 bp, Artificial Sequence
  synthetic oligonucleotide
gccaccaaaacaataatataattcacgtatgg

SEQ ID NO: 181 - DNA, 22 bp, Artificial Sequence
  synthetic oligonucleotide
cccgtttatttatttatttatt

SEQ ID NO: 182 - DNA, 23 bp, Artificial Sequence
  synthetic oligonucleotide
acccgtttatttatttttttatt

SEQ ID NO: 183 - DNA, 30 bp, Artificial Sequence
  synthetic oligonucleotide
caggaggcagttattgagaaaacagattat

SEQ ID NO: 184 - DNA, 28 bp, Artificial Sequence
  synthetic oligonucleotide
ggtaaaacagcgaaaatccttatcatgt

SEQ ID NO: 185 - DNA, 18 bp, Artificial Sequence
  synthetic oligonucleotide
attcaaccagttaataaa

SEQ ID NO: 186 - DNA, 22 bp, Artificial Sequence
  synthetic oligonucleotide
aaatattcaaccaattaataaa

SEQ ID NO: 187 - DNA, 32 bp, Artificial Sequence
  synthetic oligonucleotide
caattgttcatcaataattagcagtggtaagt

SEQ ID NO: 188 - DNA, 28 bp, Artificial Sequence
  synthetic oligonucleotide
gacaattgccctttacatatcacatgtt

SEQ ID NO: 189 - DNA, 16 bp, Artificial Sequence
  synthetic oligonucleotide
cctccttacagccttg

SEQ ID NO: 190 - DNA, 18 bp, Artificial Sequence
  synthetic oligonucleotide
ttcctccttaaagccttg

SEQ ID NO: 191 - DNA, 25 bp, Artificial Sequence
  synthetic oligonucleotide
gtgagtatttcagtgccacagtttt

SEQ ID NO: 192 - DNA, 26 bp, Artificial Sequence
  synthetic oligonucleotide
tgacaacaagttgaaggtttctcaga

SEQ ID NO: 193 - DNA, 14 bp, Artificial Sequence
  synthetic oligonucleotide
cgcgcacgttccta

SEQ ID NO: 194 - DNA, 14 bp, Artificial Sequence
  synthetic oligonucleotide
cgcgcacattccta

SEQ ID NO: 195 - DNA, 25 bp, Artificial Sequence
  synthetic oligonucleotide
cctgcatacagacaagcattgaatc

SEQ ID NO: 196 - DNA, 20 bp, Artificial Sequence
  synthetic oligonucleotide
gcctcgagtgtgctcttagc

SEQ ID NO: 197 - DNA, 19 bp, Artificial Sequence
  synthetic oligonucleotide
aaactgatttacgatttag

SEQ ID NO: 198 - DNA, 19 bp, Artificial Sequence
  synthetic oligonucleotide
aaactgatttacaatttag

SEQ ID NO: 199 - DNA, 29 bp, Artificial Sequence
  synthetic oligonucleotide
cttcaggacattgtatttacgaaaaagca

SEQ ID NO: 200 - DNA, 25 bp, Artificial Sequence
  synthetic oligonucleotide
cttacatggaattccaagctgcaaa

SEQ ID NO: 201 - DNA, 20 bp, Artificial Sequence
  synthetic oligonucleotide
aacaatctctttgatgactt

SEQ ID NO: 202 - DNA, 23 bp, Artificial Sequence
  synthetic oligonucleotide
aagaacaatctctttaatgactt

SEQ ID NO: 203 - DNA, 23 bp, Artificial Sequence
  synthetic oligonucleotide
cgtctctaggaacagccttcttg

SEQ ID NO: 204 - DNA, 25 bp, Artificial Sequence
  synthetic oligonucleotide
ccggtcctgtgtacaatgtaacttg

SEQ ID NO: 205 - DNA, 15 bp, Artificial Sequence
  synthetic oligonucleotide
cgtcgtcgtgtagct

SEQ ID NO: 206 - DNA, 17 bp, Artificial Sequence
  synthetic oligonucleotide
cacgtcgtcttgtagct

SEQ ID NO: 207 - DNA, 28 bp, Artificial Sequence
  synthetic oligonucleotide
ggacatatgaacagtgaatatgctgaga

SEQ ID NO: 208 - DNA, 40 bp, Artificial Sequence
  synthetic oligonucleotide
gtgttaaaaaaagttttgaaacattatcaattatatcagt

SEQ ID NO: 209 - DNA, 21 bp, Artificial Sequence
  synthetic oligonucleotide
cgtaagcataccttattggta

SEQ ID NO: 210 - DNA, 22 bp, Artificial Sequence
  synthetic oligonucleotide
acgtaagcataccttcttggta

SEQ ID NO: 211 - DNA, 42 bp, Artificial Sequence
  synthetic oligonucleotide
agataaaaataagcatttattaaacatttcagaccaaaaatc

SEQ ID NO: 212 - DNA, 36 bp, Artificial Sequence
  synthetic oligonucleotide
tgattatgtagtcaggccgtaattaaaattaattga

SEQ ID NO: 213 - DNA, 19 bp, Artificial Sequence
  synthetic oligonucleotide
ttgtaaataggacacatcc

SEQ ID NO: 214 - DNA, 19 bp, Artificial Sequence
  synthetic oligonucleotide
ttgtaaataggagacatcc

SEQ ID NO: 215 - DNA, 33 bp, Artificial Sequence
  synthetic oligonucleotide
ggttttctctgacaggttttatttgaagatatt

SEQ ID NO: 216 - DNA, 25 bp, Artificial Sequence
  synthetic oligonucleotide
cacatcaaggcaaacttccaatagt

SEQ ID NO: 217 - DNA, 15 bp, Artificial Sequence
  synthetic oligonucleotide
cacaagctcagtttc

SEQ ID NO: 218 - DNA, 14 bp, Artificial Sequence
  synthetic oligonucleotide
acaagcgcagtttc

SEQ ID NO: 219 - DNA, 29 bp, Artificial Sequence
  synthetic oligonucleotide
cattttacaggtaatcaagcagagtcaag

SEQ ID NO: 220 - DNA, 23 bp, Artificial Sequence
  synthetic oligonucleotide
aattgtctgctgcaggtttatgc

SEQ ID NO: 221 - DNA, 20 bp, Artificial Sequence
  synthetic oligonucleotide
ctgatttacgatttagtacc

SEQ ID NO: 222 - DNA, 21 bp, Artificial Sequence
  synthetic oligonucleotide
ctgatttacaatttagtacca

SEQ ID NO: 223 - DNA, 29 bp, Artificial Sequence
  synthetic oligonucleotide
cttcaggacattgtatttacgaaaaagca

SEQ ID NO: 224 - DNA, 25 bp, Artificial Sequence
  synthetic oligonucleotide
cttacatggaattccaagctgcaaa

SEQ ID NO: 225 - DNA, 17 bp, Artificial Sequence
  synthetic oligonucleotide
atagtgcattgattaac

SEQ ID NO: 226 - DNA, 23 bp, Artificial Sequence
  synthetic oligonucleotide
aatttaatagtgcatttattaac

SEQ ID NO: 227 - DNA, 25 bp, Artificial Sequence
  synthetic oligonucleotide
ccgtgacagagaaatgatgtagcaa

SEQ ID NO: 228 - DNA, 29 bp, Artificial Sequence
  synthetic oligonucleotide
ctacctttgagttcgttatttaacagctt

SEQ ID NO: 229 - DNA, 17 bp, Artificial Sequence
  synthetic oligonucleotide
agcctgaggtctagttt

SEQ ID NO: 230 - DNA, 17 bp, Artificial Sequence
  synthetic oligonucleotide
agcctgaggtatagttt

SEQ ID NO: 231 - DNA, 29 bp, Artificial Sequence
  synthetic oligonucleotide
gaatgtttactcatagtttttggcatgga

SEQ ID NO: 232 - DNA, 31 bp, Artificial Sequence
  synthetic oligonucleotide
ggatattaaccttacagtgtcacacatatgc

SEQ ID NO: 233 - DNA, 15 bp, Artificial Sequence
  synthetic oligonucleotide
cgtcgtcgtgtagct

SEQ ID NO: 234 - DNA, 17 bp, Artificial Sequence
  synthetic oligonucleotide
cacgtcgtcttgtagct

SEQ ID NO: 235 - DNA, 28 bp, Artificial Sequence
  synthetic oligonucleotide
ggacatatgaacagtgaatatgctgaga

SEQ ID NO: 236 - DNA, 40 bp, Artificial Sequence; identical to SEQ ID NO: 208
  synthetic oligonucleotide

SEQ ID NO: 237 - DNA, 14 bp, Artificial Sequence
  synthetic oligonucleotide
ccaccatcaacatc

SEQ ID NO: 238 - DNA, 16 bp, Artificial Sequence
  synthetic oligonucleotide
tgccaccattaacatc

SEQ ID NO: 239 - DNA, 29 bp, Artificial Sequence
  synthetic oligonucleotide
tgatcagcttgcgaaacttcaaaatattg

SEQ ID NO: 240 - DNA, 23 bp, Artificial Sequence
  synthetic oligonucleotide
cgaccagactcatttcctaacca

SEQ ID NO: 241 - DNA, 19 bp, Artificial Sequence
  synthetic oligonucleotide
tttccggtttattaacaaa

SEQ ID NO: 242 - DNA, 17 bp, Artificial Sequence
  synthetic oligonucleotide
tccggtttatgaacaaa

SEQ ID NO: 243 - DNA, 25 bp, Artificial Sequence
  synthetic oligonucleotide
gcgggtttagtcattttggttttct

SEQ ID NO: 244 - DNA, 18 bp, Artificial Sequence
  synthetic oligonucleotide
acgaacgaaccggatcgg

SEQ ID NO: 245 - DNA, 18 bp, Artificial Sequence
  synthetic oligonucleotide
aaactgctacttaaaata

SEQ ID NO: 246 - DNA, 24 bp, Artificial Sequence
  synthetic oligonucleotide
aaataaaaactgttacttaaaata

SEQ ID NO: 247 - DNA, 26 bp, Artificial Sequence
  synthetic oligonucleotide
tttttttgacaatttgtaaaccatgc

SEQ ID NO: 248 - DNA, 45 bp, Artificial Sequence
  synthetic oligonucleotide
acagaaatcagagttatattatacgttaaatttatagatatcaaa

SEQ ID NO: 249 - DNA, 18 bp, Artificial Sequence
  synthetic oligonucleotide
ttgttattttcgaaaaat

SEQ ID NO: 250 - DNA, 20 bp, Artificial Sequence
  synthetic oligonucleotide
ttttgttattttccaaaaat

SEQ ID NO: 251 - DNA, 45 bp, Artificial Sequence
  synthetic oligonucleotide
tttgatatctataaatttaacgtataatataactctgatttctgt

SEQ ID NO: 252 - DNA, 37 bp, Artificial Sequence
  synthetic oligonucleotide
tgctatgttagtgtaagaaactatgaaatattacgtt

SEQ ID NO: 253 - DNA, 18 bp, Artificial Sequence
  synthetic oligonucleotide
atgcaacatacaatgttt

SEQ ID NO: 254 - DNA, 18 bp, Artificial Sequence
  synthetic oligonucleotide
atgcaacatacgatgttt

SEQ ID NO: 255 - DNA, 36 bp, Artificial Sequence
  synthetic oligonucleotide
cgtgactgatataattgataatgtttcaaaacttgt

SEQ ID NO: 256 - DNA, 31 bp, Artificial Sequence
  synthetic oligonucleotide
atgttcatataatacacacacaaacaagatc

SEQ ID NO: 257 - DNA, 18 bp, Artificial Sequence
  synthetic oligonucleotide
atgacgatttgtttttgt

SEQ ID NO: 258 - DNA, 18 bp, Artificial Sequence
  synthetic oligonucleotide
atgacgatttgattttgt

SEQ ID NO: 259 - DNA, 36 bp, Artificial Sequence
  synthetic oligonucleotide
tatctcaaaaaaatttgtcaacattttatgttttgg

SEQ ID NO: 260 - DNA, 33 bp, Artificial Sequence
  synthetic oligonucleotide
tctctttcgatgatttctattttagcataacga

SEQ ID NO: 261 - DNA, 21 bp, Artificial Sequence
  synthetic oligonucleotide
caaactaccctttacttagat

SEQ ID NO: 262 - DNA, 22 bp, Artificial Sequence
  synthetic oligonucleotide
tcaaactaccctttacttacat

SEQ ID NO: 263 - DNA, 15 bp, Artificial Sequence
  synthetic oligonucleotide
atgggaagcgaatat

SEQ ID NO: 264 - DNA, 18 bp, Artificial Sequence
  synthetic oligonucleotide
aagatgggaaacgaatat

SEQ ID NO: 265 - DNA, 33 bp, Artificial Sequence
  synthetic oligonucleotide
aaatcatcgaaagagaaaattcaattgtcaact

SEQ ID NO: 266 - DNA, 18 bp, Artificial Sequence
  synthetic oligonucleotide
ccagcaagcagtgacgtg

SEQ ID NO: 267 - DNA, 17 bp, Artificial Sequence
  synthetic oligonucleotide
attttccccacaaaaaa

SEQ ID NO: 268 - DNA, 17 bp, Artificial Sequence
  synthetic oligonucleotide
atttttcccacaaaaaa

SEQ ID NO: 269 - DNA, 33 bp, Artificial Sequence
  synthetic oligonucleotide
acaatgtcccattggtttataaaatttacaaaa

SEQ ID NO: 270 - DNA, 28 bp, Artificial Sequence
  synthetic oligonucleotide
acagaacaaccccaatacttttaaagga

SEQ ID NO: 271 - DNA, 16 bp, Artificial Sequence
  synthetic oligonucleotide
taaaggagcaaatttt

SEQ ID NO: 272 - DNA, 16 bp, Artificial Sequence
  synthetic oligonucleotide
taaaggagcgaatttt

SEQ ID NO: 273 - DNA, 29 bp, Artificial Sequence
  synthetic oligonucleotide
ttcaagaaatacagaacaaccccaatact

SEQ ID NO: 274 - DNA, 17 bp, Artificial Sequence
  synthetic oligonucleotide
acaaccttcatcaaaag

SEQ ID NO: 275 - DNA, 17 bp, Artificial Sequence
  synthetic oligonucleotide
acaaccttcaacaaaag

SEQ ID NO: 276 - DNA, 21 bp, Artificial Sequence
  synthetic oligonucleotide
gatgatgatgacgctgagggt

SEQ ID NO: 277 - DNA, 25 bp, Artificial Sequence
  synthetic oligonucleotide
aacttctgtaaccacgtgtctatct

SEQ ID NO: 278 - DNA, 16 bp, Artificial Sequence
  synthetic oligonucleotide
cgtcttcacaagccaa

SEQ ID NO: 279 - DNA, 18 bp, Artificial Sequence
  synthetic oligonucleotide
cccgtcttcataagccaa

SEQ ID NO: 280 - DNA, 21 bp, Artificial Sequence
  synthetic oligonucleotide
ggagaaagagcttccgtcaca

SEQ ID NO: 281 - DNA, 20 bp, Artificial Sequence
  synthetic oligonucleotide
caggaagcgttcgtgtagct

SEQ ID NO: 282 - DNA, 15 bp, Artificial Sequence
  synthetic oligonucleotide
acgactcgtgaattg

SEQ ID NO: 283 - DNA, 17 bp, Artificial Sequence
  synthetic oligonucleotide
agacgactcatgaattg

SEQ ID NO: 284 - DNA, 18 bp, Artificial Sequence
  synthetic oligonucleotide
gctccagctcccgtcttc

SEQ ID NO: 285 - DNA, 21 bp, Artificial Sequence
  synthetic oligonucleotide
cttaaatcacaatcaactaat

SEQ ID NO: 286 - DNA, 22 bp, Artificial Sequence
  synthetic oligonucleotide
acttaaatcacaatcgactaat

SEQ ID NO: 287 - DNA, 19 bp, Artificial Sequence
  synthetic oligonucleotide
gcatgcaggcgtaggatct

SEQ ID NO: 288 - DNA, 29 bp, Artificial Sequence
  synthetic oligonucleotide
gactcttacaaagacaagagctcactatt

SEQ ID NO: 289 - DNA, 15 bp, Artificial Sequence
  synthetic oligonucleotide
tctgtcgcggatgtt

SEQ ID NO: 290 - DNA, 19 bp, Artificial Sequence
  synthetic oligonucleotide
taggtctgtcgtggatgtt

SEQ ID NO: 291 - DNA, 26 bp, Artificial Sequence
  synthetic oligonucleotide
gaaaaaaagtgagcaacaaaggagga

SEQ ID NO: 292 - DNA, 26 bp, Artificial Sequence
  synthetic oligonucleotide
gtgttatcttcatggttgtgttgtgt

SEQ ID NO: 293 - DNA, 20 bp, Artificial Sequence
  synthetic oligonucleotide
cttgaactatcattaaataa

SEQ ID NO: 294 - DNA, 21 bp, Artificial Sequence
  synthetic oligonucleotide
tcttgaactatcaataaataa

SEQ ID NO: 295 - DNA, 23 bp, Artificial Sequence
  synthetic oligonucleotide
actcgttcaaactaagtagcagt

SEQ ID NO: 296 - DNA, 26 bp, Artificial Sequence
  synthetic oligonucleotide
cttgccttgaaaggtatttgtctgtt

SEQ ID NO: 297 - DNA, 24 bp, Artificial Sequence
  synthetic oligonucleotide
atggtctaactattttaatacaat

SEQ ID NO: 298 - DNA, 23 bp, Artificial Sequence
  synthetic oligonucleotide
tggtctaactattttaaaacaat

SEQ ID NO: 299 - DNA, 31 bp, Artificial Sequence
  synthetic oligonucleotide
taagggtgtactttttaatttacatttttag

SEQ ID NO: 300 - DNA, 30 bp, Artificial Sequence
  synthetic oligonucleotide
ttaaaacagcaatttgacatattcatataa

SEQ ID NO: 301 - DNA, 21 bp, Artificial Sequence
  synthetic oligonucleotide
agattatcaaaacaaatggat

SEQ ID NO: 302 - DNA, 24 bp, Artificial Sequence
  synthetic oligonucleotide
aatagattatcaaaacaattggat

SEQ ID NO: 303 - DNA, 30 bp, Artificial Sequence
  synthetic oligonucleotide
ttatatgaatatgtcaaattgctgttttaa

SEQ ID NO: 304 - DNA, 29 bp, Artificial Sequence
  synthetic oligonucleotide
ggtttaatcatctacccatttctcatcct

SEQ ID NO: 305 - DNA, 24 bp, Artificial Sequence
  synthetic oligonucleotide
agctaaagataaaataaaaatgta

SEQ ID NO: 306 - DNA, 24 bp, Artificial Sequence
  synthetic oligonucleotide
agctaaagataaaataaaaatata

SEQ ID NO: 307 - DNA, 35 bp, Artificial Sequence
  synthetic oligonucleotide
tccactaaataaattctaaaataaacactaatcat

SEQ ID NO: 308 - DNA, 40 bp, Artificial Sequence
  synthetic oligonucleotide
ctatttaacactcaaaataactgcaacttaatatacatca

SEQ ID NO: 309 - DNA, 15 bp, Artificial Sequence
  synthetic oligonucleotide
aaaccacagaaaaat

SEQ ID NO: 310 - DNA, 18 bp, Artificial Sequence
  synthetic oligonucleotide
atgaaaccacataaaaat

SEQ ID NO: 311 - DNA, 25 bp, Artificial Sequence
  synthetic oligonucleotide
gcaatgcttcattagcctatgctta

SEQ ID NO: 312 - DNA, 20 bp, Artificial Sequence
  synthetic oligonucleotide
cactaggccaacacctcgtt

SEQ ID NO: 313 - DNA, 13 bp, Artificial Sequence
  synthetic oligonucleotide
acggtgaataacg

SEQ ID NO: 314 - DNA, 15 bp, Artificial Sequence
  synthetic oligonucleotide
tgacggtaaataacg

SEQ ID NO: 315 - DNA, 25 bp, Artificial Sequence
  synthetic oligonucleotide
gcatgcatcgtgatttcttaagcaa

SEQ ID NO: 316 - DNA, 35 bp, Artificial Sequence
  synthetic oligonucleotide
ccaaacactcaaattataatttttggttaattcca

SEQ ID NO: 317 - DNA, 16 bp, Artificial Sequence
  synthetic oligonucleotide
tgtgtggaaacaaaag

SEQ ID NO: 318 - DNA, 16 bp, Artificial Sequence
  synthetic oligonucleotide
tgtgtggaagcaaaag

SEQ ID NO: 319 - DNA, 30 bp, Artificial Sequence
  synthetic oligonucleotide
gattttccagataattgaacgaaccttctc

SEQ ID NO: 320 - DNA, 26 bp, Artificial Sequence
  synthetic oligonucleotide
acacacacacacatatcttcccaaaa

SEQ ID NO: 321 - DNA, 17 bp, Artificial Sequence
  synthetic oligonucleotide
acatcttcagcttttca

SEQ ID NO: 322 - DNA, 19 bp, Artificial Sequence
  synthetic oligonucleotide
agacatcttcagtttttca

SEQ ID NO: 323 - DNA, 25 bp, Artificial Sequence
  synthetic oligonucleotide
gtatttgcatgtggtcagtgtaacc

SEQ ID NO: 324 - DNA, 26 bp, Artificial Sequence
  synthetic oligonucleotide
caaggttggcagcaatcataatttca

SEQ ID NO: 325 - DNA, 21 bp, Artificial Sequence
  synthetic oligonucleotide
ctgattaatcatgaactttta

SEQ ID NO: 326 - DNA, 21 bp, Artificial Sequence
  synthetic oligonucleotide
ctgattaatcatgaattttta

SEQ ID NO: 327 - DNA, 33 bp, Artificial Sequence
  synthetic oligonucleotide
tcctagaattaatcatgaacttttttttaacgt

SEQ ID NO: 328 - DNA, 26 bp, Artificial Sequence
  synthetic oligonucleotide
gaggttgatctagatagagcagaaga

SEQ ID NO: 329 - DNA, 19 bp, Artificial Sequence
  synthetic oligonucleotide
caaatgtagccctattact

SEQ ID NO: 330 - DNA, 19 bp, Artificial Sequence
  synthetic oligonucleotide
caaatgtagccgtattact

SEQ ID NO: 331 - DNA, 33 bp, Artificial Sequence
  synthetic oligonucleotide
cttaaggttttacaagctagactgattatgaca

SEQ ID NO: 332 - DNA, 25 bp, Artificial Sequence
  synthetic oligonucleotide
agagatccaagcttgggaaacatag

SEQ ID NO: 333 - DNA, 21 bp, Artificial Sequence
  synthetic oligonucleotide
caaataaaataacggctaaat

SEQ ID NO: 334 - DNA, 22 bp, Artificial Sequence
  synthetic oligonucleotide
ccaaataaaataacgactaaat

SEQ ID NO: 335 - DNA, 25 bp, Artificial Sequence
  synthetic oligonucleotide
tgcgcgatacttatgactggttaac

SEQ ID NO: 336 - DNA, 31 bp, Artificial Sequence
  synthetic oligonucleotide
cggactctataacccacttctaaaaacaata

SEQ ID NO: 337 - DNA, 24 bp, Artificial Sequence
  synthetic oligonucleotide
attaataactttttttaataattg

SEQ ID NO: 338 - DNA, 25 bp, Artificial Sequence
  synthetic oligonucleotide
aattaataactttttttaatgattg

SEQ ID NO: 339 - DNA, 31 bp, Artificial Sequence; identical to SEQ ID NO: 299
  synthetic oligonucleotide

SEQ ID NO: 340 - DNA, 30 bp, Artificial Sequence; identical to SEQ ID NO: 300
  synthetic oligonucleotide

SEQ ID NO: 341 - DNA, 15 bp, Artificial Sequence
  synthetic oligonucleotide
attggtcagaaagac

SEQ ID NO: 342 - DNA, 20 bp, Artificial Sequence
  synthetic oligonucleotide
atcttattggtaagaaagac

SEQ ID NO: 343 - DNA, 29 bp, Artificial Sequence
  synthetic oligonucleotide
gggaggtaaagttgagttctttatggaat

SEQ ID NO: 344 - DNA, 24 bp, Artificial Sequence
  synthetic oligonucleotide
ctacttatccaccggacaaaagct

SEQ ID NO: 345 - DNA, 19 bp, Artificial Sequence
  synthetic oligonucleotide
tggcttgaagacatagaca

SEQ ID NO: 346 - DNA, 19 bp, Artificial Sequence
  synthetic oligonucleotide
tggcttgaagacataaaca

SEQ ID NO: 347 - DNA, 27 bp, Artificial Sequence
  synthetic oligonucleotide
atgccctcatttgtaagatatcaagca

SEQ ID NO: 348 - DNA, 30 bp, Artificial Sequence
  synthetic oligonucleotide
gagaaaatgagtaagaagatcaaagcttca

SEQ ID NO: 349 - DNA, 20 bp, Artificial Sequence
  synthetic oligonucleotide
aagaacaagaaatggagtct

SEQ ID NO: 350 - DNA, 22 bp, Artificial Sequence
  synthetic oligonucleotide
agaagaacaagaaatagagtct

SEQ ID NO: 351 - DNA, 24 bp, Artificial Sequence
  synthetic oligonucleotide
caagcagtagactcaacagaatgc

SEQ ID NO: 352 - DNA, 28 bp, Artificial Sequence
  synthetic oligonucleotide
cactgaccacatgcaaatactctacata

SEQ ID NO: 353 - DNA, 16 bp, Artificial Sequence
  synthetic oligonucleotide
cctgttgtgatgcatc

SEQ ID NO: 354 - DNA, 18 bp, Artificial Sequence
  synthetic oligonucleotide
tgcctgttgtaatgcatc

SEQ ID NO: 355 - DNA, 36 bp, Artificial Sequence
  synthetic oligonucleotide
agcgtagttactttccatactgaatcaatataatag

SEQ ID NO: 356 - DNA, 32 bp, Artificial Sequence
  synthetic oligonucleotide
aatggtttccttttgatgatttttcttgaact

SEQ ID NO: 357 - DNA, 18 bp, Artificial Sequence
  synthetic oligonucleotide
tgaacgagtacgtgcctg

SEQ ID NO: 358 - DNA, 16 bp, Artificial Sequence
  synthetic oligonucleotide
aacgagtgcgtgcctg

SEQ ID NO: 359 - DNA, 38 bp, Artificial Sequence
  synthetic oligonucleotide
ccatactgaatcaatataataggttttctatcaaatgc

SEQ ID NO: 360 - DNA, 30 bp, Artificial Sequence
  synthetic oligonucleotide
cacctagaatagaatggtttccttttgatg

SEQ ID NO: 361 - DNA, 21 bp, Artificial Sequence
  synthetic oligonucleotide
atggttctttatgctcatagt

SEQ ID NO: 362 - DNA, 21 bp, Artificial Sequence
  synthetic oligonucleotide
atggttctttatgctcaaagt

SEQ ID NO: 363 - DNA, 27 bp, Artificial Sequence
  synthetic oligonucleotide
aggttgttcatttccttaatccacaca

SEQ ID NO: 364 - DNA, 25 bp, Artificial Sequence
  synthetic oligonucleotide
aacttctgtaaccacgtgtctatct

SEQ ID NO: 365 - DNA, 16 bp, Artificial Sequence
  synthetic oligonucleotide
cacattcagacaaact

SEQ ID NO: 366 - DNA, 20 bp, Artificial Sequence
  synthetic oligonucleotide
ctttcacattcagacagact

SEQ ID NO: 367 - DNA, 25 bp, Artificial Sequence
  synthetic oligonucleotide
ccaatcacagctcagtaatgaacca

SEQ ID NO: 368 - DNA, 21 bp, Artificial Sequence
  synthetic oligonucleotide
tagcaagttgttccttaatta

SEQ ID NO: 369 - DNA, 20 bp, Artificial Sequence
  synthetic oligonucleotide
agcaagttgttccctaatta

SEQ ID NO: 370 - DNA, 41 bp, Artificial Sequence
  synthetic oligonucleotide
ctcaatgaatttactaaattactgtattttgtaatcttcgt

SEQ ID NO: 371 - DNA, 25 bp, Artificial Sequence
  synthetic oligonucleotide
cagactcttttgccacttctcgata